(19) United States
(12) Patent Application Publication
(10) Pub. No.: US 2026/0009034 A1
(43) Pub. Date: Jan. 8, 2026

(54) NOVEL THERAPY

(71) Applicant: LINNANE PHARMA AB, Malmo (SE)

(72) Inventors: Catharina SVANBORG, Malmo (SE); Caterina CAFARO, Lund (SE)

(21) Appl. No.: 19/331,956

(22) Filed: Sep. 17, 2025

Related U.S. Application Data
(62) Division of application No. 16/341,248, filed on Apr. 11, 2019.

(30) Foreign Application Priority Data
Oct. 14, 2016 (GB) 1617470.8

(51) Int. Cl. C12N 15/113; A61K 35/74; A61K 38/00; C07K 14/245; C12N 1/20

(52) U.S. Cl. CPC C12N 15/1135; A61K 35/74; C07K 14/245; C12N 1/20; A61K 38/00; C12N 2310/141

(57) ABSTRACT

A therapeutic agent comprising a bacteria which expresses an inhibitor of MYC activity or a extract or product obtainable therefrom, for use in for use in the prevention or treatment o disease wherein MYC levels are elevated, such as cancer. Inhibitors of MYC activity comprising a factor obtainable from bacteria are novel and form a further aspect of the invention.

This application is a divisional application of U.S. Ser. No. 16/341,248 filed Apr. 11, 2019, which was a U.S. National Stage Application of International Application No. PCT/IB2017/056369, filed Oct. 13, 2017, which claims the benefit of, and priority to, Great Britain Patent Application No. 1617470.8, filed Oct. 14, 2016, the entire contents of which are hereby incorporated by reference in their entirety.

This application contains a Sequence Listing that has been submitted in ST.26 format via Patent Center and is hereby incorporated by reference in its entirety. The ST. 26 copy, created on Sep. 16, 2025, is named 051875-858451 Sequence listing.xml, and is 10,173 bytes in size.

The present invention relates to entities for suppressing MYC activity, to the use of these entities in treating diseases such as cancer where MYC activity is undesirable.

Myc (c-Myc) is a regulator gene that codes for the MYC transcription factor. The protein encoded by this gene is a multifunctional, nuclear phosphoprotein that plays a role in cell cycle progression, apoptosis and cellular transformation.

However, Myc is recognised as being a key oncogene. A mutated version of Myc which causes Myc to be constitutively expressed is found in many cancers. This leads to the unregulated expression of many genes, some of which are involved in cell proliferation, and results in the formation of tumours. Malfunctions in Myc have been found in cancers such as cervical cancer, colon cancer, breast cancer, lung cancer and stomach cancer for example. Thus, Myc is viewed as a clear target for anti-cancer drugs.

The applicants have been studying susceptibility to infection and found that this differs markedly between individuals. Rare primary immuno-deficiencies predispose children to infection and individuals with acquired immuno-deficiencies run an increased risk of viral, bacterial and parasitic infections. Host resistance is classically impaired by mutations that directly affect the antimicrobial effector functions of lymphocytes, immunoglobulin-producing cells or inflammatory cells. In addition, the susceptibility to infection is determined by the efficiency of host gene expression, with a clear distinction between asymptomatic and symptomatic infections. Attenuating TLR4 promoter polymorphisms often accompany asymptomatic bacteriuria (ABU) and T1r4^{-/-} mice develop asymptomatic bacteriuria rather than systemic infection. In contrast, attenuating IRF3 promoter polymorphisms are associated with susceptibility to acute pyelonephritis (APN) and Irf3^{-/-} mice develop acute, septic infections accompanied by massive tissue damage. Tissue pathology is driven by the closely related transcription factor Irf7, which regulates the expression of proinflammatory, tissue destructive genes. Attenuating IRF7 promoter polymorphisms are negatively associated with APN susceptibility in children but specific molecular determinants of pathology, downstream of Irf7, remain to be defined.

Around 10% of expressed human genes are involved in transcription and mechanisms of transcriptional regulation have been extensively characterized. Innate immunity and inflammation is regulated by multiple gene sets that control transcriptional “modules” and functional programs (Medzhitov and Horning, 2009). The constitutively expressed Class I transcription factors, are activated by post-translational modifications during the initial phase of innate immune activation and include IRF-3 and NF-κB. The Class II category, which requires de novo synthesis, regulates subsequent waves of gene expression and may in some cases, stably reprogram transcription. This category includes IRF-7 and different c-Fos and c-Jun related C/EBP isoforms, which also interact with CREB and NF-κB, in infected cells.

### SUMMARY OF THE INVENTION

During an investigation into the inherent, gene expression differences distinguishing highly infection-prone individuals from asymptomatic bacterial carriers, the applicants have surprisingly identified c-MYC as a key transcriptional regulator. Specifically, the applicants identified MYC and MYCN as major regulators of mucosal innate immune responses, downstream of IRF7. By genome-wide RNA sequencing, an “immunological disequilibrium” was detected in patients prone to acute pyelonephritis (APN), associated with an elevated MYC and TCR expression but an unresponsive innate immune system. In contrast, asymptomatic carriers showed an innate immune response profile with modest activation of IRF7-dependent genes and low MYC expression.

The genetic basis for this discrepancy was examined in the murine pyelonephritis model, where Irf7 was identified as a key regulator of MYC expression, with pronounced inhibition of MYC in Irf7^{-/-} mice that were protected from pathology.

According to the present invention there is provided a therapeutic agent comprising a bacteria which expresses an inhibitor of MYC activity or a extract or product obtainable therefrom, for use in for use in the prevention or treatment of disease wherein MYC levels are elevated.

The applicants have found that certain bacteria, in particular certain uropathogenic bacteria inhibit MYC activity in a host, and that this is caused by expression of a bacterial factor, which appears to be secreted by the bacteria, and thus is present in supernatants. Thus in a particular embodiment, the therapeutic agent comprises an extract of product obtainable from bacteria, which comprises or is derived from a supernatant produced during culture of the bacteria.

Furthermore, they have identified that the mechanism by which the bacteria and in particular the factor obtainable from the bacteria exerts its effect. The identified mechanism has been found to comprises a direct inhibitory effect of bacteria on mucosal MYC expression, due to a new class of virulence factors encoded on PAI I in uro-pathogenic E. coli strains. The findings suggest a scenario where mucosal MYC expression is regulated downstream of Irf3 and Irf7, which are major determinants of disease severity and susceptibility in infected kidneys and that MYC is targeted by specific bacterial genes on pathogenicity islands that are shared by uropathogenic E. coli strains.

Furthermore, the applicants have found that MYC may be degraded by a second strategy, in which activation of CK1α1 And IFNβ dependent mechanisms of phosphorylation and proteasomal degradation by the host is triggered.

The isolated factors are novel and so these form a further aspect of the invention.

Thus according to a second aspect of the present invention there is provided an inhibitor of MYC activity comprising a factor obtainable from a bacteria. In particular, the bacteria is a pathogenic bacteria, such as a uropathogenic E. coli. Particular examples of such uropathogenic E. coli is selected from CFT073 and 536.

As used herein, the expression ‘inhibitor of MYC activity’ refers to any entity which controls, regulates or limits the activity of the MYC transcription factor, in particular in vivo in mammals. This may be achieved for example by direct inhibition of the MYC protein, by downregulation of the c-Myc gene in vivo, or by interfering with any pathway in which MYC is involved.

Bacteria can evolve by modification of the pre-existing genome sequence, acquisition or loss of genetic information. Gene acquisition by horizontal gene transfer, involving mobile genetic elements like plasmids or genomic islands (GEIs), increases the capacity of bacteria to evolve and adapt to new environments.

The pathogenic properties of bacteria are correlated to the expression of virulence- or disease-associated factors, that are usually absent in non-pathogenic strains. These virulence factors are encoded in so called pathogenicity islands (PAIs), genomic islands of 10-100 kb acquired by horizontal gene transfer. PAIs, usually associated to tRNA genes, are often flanked by direct repeat sequences and have a G+C content that differs from the bacterial “core genome” (Gal-Mor, O. and B. B. Finlay (2006). Cell Microbiol 8(11): 1707-1719).

PAIs comprise different factors involved in genetic mobility (like transposases and integrases), bacterial adherence factors (P-related pili, S-fimbriae), siderophores, exotoxins (like α-haemolysin) and genes related to bacterial invasion.

In a particular embodiment, the inhibitor which comprises a protein expressed by a gene found in a PAI I or PAI II region of the genome of said bacteria, or a combination thereof. In a particular embodiment, the inhibitor comprises a protein expressed by a gene found in a PAI 1 region of the genome of the uropathogenic Escherichia (E.) coli strain 536.

PAI I consists of 76,843 bp and encodes for alpha-haemolysin gene cluster, fimbriae and adhesins as well as many unidentified proteins (Dobrindt, U., et al. (2002). Infect Immun 70(11): 6365-6372). Specifically, in PAI I there are 76 genes and 4 pseudo genes. Of the 76 genes, 50 are still classified as “unnamed” in the pathogenicity island database (www.paidb.re.kr) and just 16 genes were tested for their virulence properties, leaving all the others to be tested.

All the unnamed genes or the relative proteins are homologous to genes or proteins from other bacterial species. Therefore, inhibitors may be obtainable from a wide range of bacterial species, including for example, Streptococcus, Pseudomonas, Shigella, Campylobacter, and Salmonella species.

By analysing individual genes or gene products, located in PAI I, a skilled person would be able to identify the specific factor or factors required to regulate or inhibit c-MYC. Such analysis may be carried out using various methods, including for example, methods illustrated hereinafter in which E. coli 536 mutants, each lacking sequential PAI I fragments, are used for example to assess c-MYC regulation in vitro, in kidney epithelial cells or to analyse the MYC response in a murine model of kidney infection.

Once identified, the active factor or factors may be isolated from the pathogenic bacteria. However, in a particular embodiment, they may be produced in isolated form, either synthetically or using recombinant DNA production to avoid use of pathogenic bacteria. They may then be used in therapy as explained further hereinafter.

In a particular embodiment, the inhibitor may comprise the product of a leuX gene of a bacteria such as E. coli. Such genes encode a suppressor tRNA that inserts leucine at the amber codon. Alternatively, the inhibitor may comprise a product of an hly gene, such as an hly I or hly II gene of E. coli. Such products may comprise the toxin, Listeriolysin O.

In a particular embodiment, the inhibitor is a bacterial ABC transporter protein or an active fragment thereof.

ABC transporter proteins are integral membrane proteins which comprise ATP binding cassette transporters. They are characterized by two nucleotide-binding domains (NBD) and two transmembrane domains (TMDs). ATP hydrolysis on the NBD drives conformational changes in the TMD, resulting in alternating access from inside and outside of the cell for unidirectional transport across the lipid bilayer. Common to all ABC transporters is a signature sequence or motif, LSGGQ, that is involved in nucleotide binding.

Both importing and exporting ABC transporters are found in bacteria. In a particular embodiment, the inhibitor comprises a protein encoded on the 2.5 region of E. coli 536 of SEQ ID NO 1:

(SEQ ID NO: 1)
ATCAGCATGGCAGTCAGTCTGCGGGCATAATTTTTATCGTGTGTTTTATG
GATGGCTTTCTGCATCAGGTGTCGTTCGTCACGGGAAATTGGTGCTATGA
TCGGCATTGCTCAGTTCGGTTGGTGATTTGTTTTGATTTGGCGATTGATC
AGAGCGCACAATTCGGGCTGAGTACCCTCAAAGTGATCTACTATTCCGCG
CAGCTATTTAGCGCATTACACTGCTAAATGACTAACGCATCCCTGTCAAT
ACTGCTCTCGCTGTATGCTTACGCTGCAGAAGCCATGACACATTTTCTTA
CCTATCATCGGCACCCATAAAGCAACAGTGTTATACTATAACAATTTTGA
TGGGGGATACTGTGGTCAGTCTTTATGCTTTTTCCGCAATAGCCCGACTG
GGTATCGTCGGTGTGCTGCTGGTACTGTTATGGACTCTTATTTCTTGGGC
GGTGGTACTGGCATGATCGCATTACATTCACTGGCATTTGGTTATTCGGG
ACAGCACCCGCTGGGTACGCTGGATGGATGTTTTGACACCGGTTCACTGA
CTGCCATTATCGGAGCGAATGGCACAGGTAAGTCCACCTTGCTTAAAACG
TTGGCCGGTCTGCTGCCACCCCTGGGGGGTTGCTTCTGTATGGTACCGCA
GGGGCAACGCCAGTTAGGTTATTTGCCCCAATTGACTGAATTTGATCGCC
AGTTTCCACTGAGTGTTAACGATCTGGTGCTGATGGGATGTATTCCACAC
AGCGGGATGTTCGGTCGTATTTCCTGTCTATGGCGAAAAAAAGCTATCGA
AGCGCTGGATACCGTCGGTATGACAGAATTTTCGCAGATGCACATCGGCA
CACTTTCCGGTGGTCAGTTACAGCGCGTACTGTTCGCACGACTGCTGGTG
ATGCAGCCGTCGGTTATTCTGCTGGATGAACCATTTACTGGCATTGATGT
TCAGACTATCCGTACTCTGCTGGTTGTTATTCGGCAGCTACATCTGGAAG
GACGTACAATTCTTGCGGTGTTGCATGACATGGAACAGGTAGAAAAATAC
TTTTCTCATGTACTGATGCTCAGTGCGGAAGGCCACCGATGGGGGAGAAG
TGCCGATATACTCCATTCCCTCACGGCTGCAGTCACGCCACCGCAGCAGG
GGCTGCTGCCATGATGTTACTGCACCTGTTGTGTGAGCCATTTGGCGATT
TCGGTTTTATGCGTCGGGCACTGGTCGGATGTCTGGCGCTGACCCTGAGT
GCCGCGCCACTTGGCTGTTTTTTGCTACTGCGCCGTATGAGCCTGATCGG
TGATGCACTGTCCCATGCGGTGTTGCCGGGAGTAGCAATTGGCTACCTGG
TATCGGGCATGTCGCTGGTTGCGATGGGAGTCGGTGGCTTCATTGCCGGG
CTGTCTGTGGCGATGTTGTCTGGTGTGGTCAGCCGCCGCACTGGATTGAG
AGAAGACGCCAGTTTCGCTGGGTTTTACCTCGGCTCGCTGGCACTTGGCG
TCACCCTGGTCTCTTTGCGTGGCTCCAGTGTCGACCTGCTGCATGTGCTG
TTCGGTTCCATTCTGGCTATTGACGCCAATGCTCTGATCACTATCGGCAT
AATTAGTTCCGGTTCAGTTCTGGTACTGGCGTTGATCTACCGGGTGCTGG
TGATTGAGTCGTTTGATGTCACTTTCCTGAAAGTTCTGTCACGTCGGTCG
CGGGCGCTGATCCACTGCCTGTTTTTGTCAATGGTGGTACTCAATCTGGT
GGCTGGTTTCCAGCTACTTGGCACCCTGATGACAGTCGGTATTATGATGT
TACCGGCTGCCAGCGCGCGTTTCTGGAGTCAACGTCTGTCCATCATGTTG
TTAGCGGCAGTGGGTATTGGTACCTGCGCCAGTCTGGTTGGACTGACCTG
GTCTTATTACGCTGATTTGCCTGCAGGCCCTGCGGTCATCCTCACGAGCA
CTCTGTTTTTCTGCTTTTCAGTTCTGTTTGGCTCTAATGGCGGCATGTTA
TGCAGATGTCGCTGACAAGAAGTGTTTTTACACATAGCAACGAGATGAAG
GAGGAAAGATGAAACGGTCCATTTTGGTTGTGGCACTGTCCAGCCTACTA
GTAAGCCCACTGGTGATTGCAAAAGAGTTGAATGTGGTGGCAAGTTTCTC
GGTACTGGGCGATATGGTCAGCCAGATTGGCGGCCCATACGTTCATGTCA
CTGATCTGGTGCAACCGGATGGTGATCCACATGAATTTGAACCGTCGCCG
AAAGACAGCAAAACGCTGGCACAGGCGGATGTTGTCTTTGTCAATGGATT
GGGACTGGAGGGATGGCTGGACCGTCTGATGAAAGCCTCTGGCTACAGAG
GTGAGGTAATTACCGCGTCTAACGGAATAGATACACTGAAAATGAAAGAG
GATGGGACCACCATCACTGATCCGCACGCCTGGAACAGCATGAAAAACGG
TATTGTCTATGCTCACAACATTGTCAATGGCCTGAGCAAAGCAGACCCGG
AGCACGCCAGCGATTATCGAAAACAGGGTGACAGCTATATCCAGCAGTTG
CAGCAACTGGATAATTACGCCACGCAGACGTTTGCTGCCATTCCAAGAGA
AAAACGTAAAGTACTGACCAGTCATGATGCGTTTGGGTATTTCGCTGCCG
CCTACGGGGTTCGCTTTCTGTCGCCAGTGGGGTACTCCACTGAGTCGGAA
GCTAGCAGTAAAAATGTGGCGAAACTGATTAATCAAATTAAGAGAGAGCA
CGTAAAATTGTACTTTATCGAAAACCAGACCGATCCCCGTTTGGTGAAAC
AAATCGCCAATGCCAGTGGTGCACAAGCGGGAGGAGAGCTTTATCCGGAA
GCACTTACCGACAGTAGCGGCCTCGCTGCTACCTACACGGCAGCGTTCAA
ACACAATGTCGATACCCTCGCTGCTGGCATGAAGTAAGAGAGTTGGCAAA
AAAGTAAGTAGATATCAGGTTGCCCCATATCATCAGGTAACCTGTGACAC
TTCTCTTGCCAGAGCGCCATGTTCCAGCGCCTCACTCACTATCTGCTTAT
CATCCGACTTTCCCTCATACCTGTCTCGATTATAAAGATAAAACGATGAT
GCCCGGGTTCATTGTGCGAAGGCATGGCATATTTGTTCCCGGTGTCGTCA
GACAGCATCATTCGACTCTCCATCTGCTGTCTGACCAGACAAAAGATGGC
CTTGTTTGCCGCGGTGGAAATGGAGTGCCGTCTGTTCTTTATTTATTGTT
TATGTTATGTTATAGCATATAAAAGAGTATTGTTTGGCATCTGTAACTTA
TTGAGAAGGCAAACTGCAGAGTGGTTAATGCAGTAATTGTATTAGCTAAA
ATTTTGTTAATCAAAATTTGTTTGATTGTTAATG.

As used herein, the term ‘fragment’ refers to any portion of the given amino acid sequence which will shows MYC inhibitory activity. Fragments may comprise more than one portion from within the full-length protein, joined together. Portions will suitably comprise at least 5 and preferably at least 10 consecutive amino acids from the basic sequence.

Suitable fragments will include deletion mutants comprising at least 10 amino acids, for instance at least 20, more suitably at least 50 amino acids in length or analogous synthetic peptides with similar structures. They include small regions from the protein or combinations of these. Depending upon the nature of the bacteria and the disease to be treated, it may be possible to use a bacteria which expresses an inhibitor of MYC activity as described above for use in the prophylaxis or treatment of disease wherein MYC levels are elevated. In this case, where the bacteria is a pathogenic bacteria, an attenuated form of the bacteria (e.g. attenuated Salmonella species) may be used provided it retains the MYC inhibitory activity. In a particular embodiment, the bacteria is uropathogenic bacteria, including for example, E. coli such as E. coli 536 or CFT073 which are attenuated.

Attenuation in this instance may be effected using conventional methods including for example, by heat treatment or chemical treatment for example with formaldehyde, acetone, phenol or propiolactone, as well as by cuture in unfavourable conditions and/or by the use of recombinant DNA technology to remove or inactivate genes, in particular virulence genes.

Alternatively however, the bacteria may comprise a recombinant bacteria, and in particular a non-pathogenic bacteria such as an ABU strain, which has been engineered so as to express an inhibitor as described herein.

For administration to patients, the inhibitor or bacteria is suitably administered in the form of a pharmaceutical composition, which further comprise a pharmaceutically acceptable carrier. Such compositions form a further aspect of the invention.

Suitable pharmaceutical compositions will be in either solid or liquid form. They may be adapted for administration by any convenient route, such as parenteral, oral or topical administration or for administration by inhalation or insufflation. The pharmaceutical acceptable carrier may include diluents or excipients which are physiologically tolerable and compatible with the active ingredient.

Parenteral compositions are prepared for injection, for example either subcutaneously or intravenously. They may be liquid solutions or suspensions, or they may be in the form of a solid that is suitable for solution in, or suspension in, liquid prior to injection. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

Oral formulations will be in the form of solids or liquids, and may be solutions, syrups, suspensions, tablets, pills, capsules, sustained-release formulations, or powders. Oral formulations include such normally employed excipients as, for example, pharmaceutical grades of mannitol, lactose, starch, magnesium stearate, sodium saccharin, cellulose, magnesium carbonate, and the like.

Topical formulations will generally take the form of suppositories or intranasal aerosols. For suppositories, traditional binders and excipients may include, for example, polyalkylene glycols or triglycerides; such suppositories may be formed from mixtures containing the active ingredient.

In yet a further aspect, the invention provides a method of treating or preventing a disease in which MYC levels are elevated, said method comprising administering to a patient in need thereof, an effective amount of an inhibitor as described above, or a bacteria which expresses said inhibitor, or a pharmaceutical composition comprising either of these.

The amount of inhibitor administered will vary in accordance with normal clinical practice, and will depending upon factors such as the nature of the reagent being used, the size and health of the patient, the nature of the condition being treated etc. in accordance with normal clinical practice. Typically, a dosage in the range of from 1 μg-50 mg/Kg for instance from 2-20 mg/Kg, such as from 5-15 mg/Kg would be expected to produce a suitable effect.

In particular, the disease in which MYC levels are elevated and which is therefore susceptible to treatment using the method of the invention is cancer. Examples of such cancers may include cervical cancer, colon cancer, breast cancer, lung cancer, stomach cancer, kidney cancer, bladder cancer, bowel cancer, mouth cancer or cancer of the gastrointestinal track. In a particular embodiment, the cancer may be associated with a mucosal or epithelial surface, such as cervical cancer, lung cancer, kidney cancer, bladder cancer, bowel cancer, mouth cancer or cancer of the gastrointestinal track.

The pleiotropic MYC transcription factors regulate cellular life and increased MYC activity is associated with oncogenic transformation. To reiterate, the applicants have made the unexpected observation that the c-MYC family of transcription factors regulate innate immune responses to bacterial infection in a tissue specific manner, involving the mucosal barrier. They first detected elevated MYC transcript levels in children prone to acute pyelonephritis, which is a common and severe bacterial infection of the kidneys. The applicants also identified Irf3 and Irf7 as transcriptional regulators of Myc expression in the murine acute pyelonephritis model and characterized the profile of c-MYC dependent genes involved in tissue repair. However in accordance with the invention, they have shown that C-MYC expression is inhibited by uropathogenic E. coli infection, through a new class of bacterial virulence factors.

These findings are unexpected; effects of c-MYC on host susceptibility to bacterial infection have not been described previously. Different mycobacterial species induce MYC expression via ERK1/2 and JNK, leading to TNF-α and IL-6 activation and suppression of intracellular mycobacterial growth and a MYC-dependent global metabolic transcriptome has been detected in activated, primary T lymphocytes. In addition, as c-MYC plays an important role in T cell priming during viral or a bacterial infection, effects on the specific immune response have been extensively documented. Wang et al. (2011) Immunity, 2011. 35(6): p. 871-82 showed that T cell activation leads to increased glucose, glutamine, and mitochondrial oxidative metabolisms that are essential for growth and proliferation.

In addition to MYC, MYCN was differentially activated in the APN and primary ABU patients. Previously, the amplification of MYCN in neuroblastoma cell lines has been shown to affect both immune cell recruitment and chemokine expression and an inverse correlation between MYCN RNA levels and monocyte chemoattractant protein-1/CC chemokine ligand 2 (MCP-1/CCL2) has been observed. CCL2 is a prototypic inflammatory chemokine and functions as a major chemoattractant for monocytes, both CD4 and CD8 effector-memory α/β T cells, γ/δ T cells and NKT cells. Overexpression of MYCN has therefore been linked to repressed expression of monocyte chemoattractant protein-1/CC chemokine ligand 2 (MCP-1/CCL2). The MYC/MYCN activation in the APN patients and inhibition in the primary ABU patients might therefore constibute to the difference in transcriptional activity in the two patient groups.

MYC was inhibited during the early stage of infection and c-MYC dependent genes showed an inhibitory profile. More than 1000 genes were regulated and regulated genes were mostly suppressed. In Irf3^{-/-}, mice, where bacteria remained, there was a transition from inhibition to activation of about 300 genes. This transition did not occur in Irf7^{-/-}, mice, suggesting that the MYC response in regulated by Irf7 in the Irf3^{-/-} background. These results also suggest that MYC acts downstream of the IRFs. Further supported by activation of IRF7 in the ABU group, but increase in MYC in the APN group, where IRF7 was unchanged compared to the control.

Acute pyelonephritis is accompanied by a strong mucosal inflammatory response, which spreads from the site of infection to systemic compartments, causing fever, elevated acute phase reactatns and urosepcsis in about 30% of the cases. The mucosal innate immune response to UPEC is controlled by TLR4 through the TRIF and TRAM adaptors. Downstream signaling involves MAP kinases, resulting in P38 and CREB-dependent activation of IRF3/IRF7. The transcription factors IRF-3 and IRF-7 form a regulatory node that controls transcription in response to uropathogenic E. coli infection, through associated downstream network. Disruption of the interdependence of these two transcription factors was recently found to determine disease outcome, as IRF-7 regulated a late, destructive inflammatory response that caused severe disease in mice, lacking the counter-balancing beneficial effect of IRF-3. Importantly, Irf7^{-/-} mice were protected from pathology and liposomal delivery of Irf7 siRNA prevented disease in susceptible, Irf3^{-/-} mice. While these transcription factors have been shown to regulate the susceptibility to APN; the regulation by IRF3/IRF7 of bacterial clearance and induction or resolution of tissue inflammation has not been identified. The applicants have identified Irf3/7 as transcriptional regulators of c-MYC in the mucosal compartment, suggesting that essential antibacterial effector functions may be regulated in a c-MYC-dependent manner.

Irf7 is expressed in the renal mucosa and is regulated by mucosal infection at this site. C-MYC showed a similar expression pattern and was regulated locally, in response to infection. In addition, the applicants obtained genetic and therapeutic evidence that MYC requires Irf7 activation, during the later stages of infection, which determine pathology. C-MYC expression was inhibited in Irf7^{-/-} mice and the response to infection was abrogated by treatment of susceptible Irf3^{-/-} mice with Irf7^{-/-} specific siRNA, suggesting that MYC is expressed in tissues where Irf7 is activated and that the restriction of MYC to the renal pelvic epithelium is defined by the need for Irf7 to initiate transcription. These findings provide a model to resolve why MYC can regulate the local innate immune response without significant involvement of the Tcell dependent, specific immune system. The results also identify tissue specific gene expression as an interesting strategy to solve the problem of transcription factor specificity and redundancy.

The present study identifies new class of bacterial virulence factors, which act by regulating c-MYC in infected host cells. MYC expression in the renal pelvis was inhibited by uropathogenic E. coli and by genome sequencing and mutation strategies, we localised the MYC inhibitory activity to chromosomal gene clusters on PAI I in E. coli 536, a classical uropathogen where the structure and regulation of virulence genes in uropathogenic E. coli was characterized. These findings are consistent with recent obervations, suggesting that bacterial virulence factors act as direct regulators of host gene expression. We recently proposed that P fimbriae serve as IRF7 agonists in the kidneys as a P fimbriated strain E. coli 83972pap caused a rapid and sustained change in gene expression, with activation of IRF7 and IRF7-dependent genes. In contrast, very few genes were activated, after inoculation with the non-fimbriated wt variant of this strain, which does not express functional P fimbriae, due to multiple point mutations in the pap gene cluster. The present study adds MYC to the list of bacterial targets and show that regulation of MYC during infection regulates virulence and disease severity in the urinary tract.

Normal quiescent human T cells express low levels of steady-state c-myc mRNA as a result of low constitutive promoter utilization, a block to transcriptional elongation within the gene, and rapid degradation of c-myc mRNA in the cytoplasm. Following the activation of the T cell receptor (TCR)/CD3 complex, quiescent T cells are induced to express more c-myc mRNA [30], indicating a potential role of MYC in the APN phenotype. By RNA sequencing, we identified major differences in immune activation between patients with APN and ABU. c-MYC expression was activated in the APN group, as was the expression of T cell receptor genes but Irf7 was not regulated. In contrast, the ABU group had low c-MYC and TCR expression levels but moderate activation of IRF7 and genes involved in neutrophil function and innate immunity. Importantly, the samples were obtained during an infection-free interval of at least one year, suggesting that these differences were determined by the basic transcriptional “persona” of individuals, potentially determining who will develop these infections.

Pleiotropic transcription factors such as MYC and SRC aquire mutations and can turn into oncogenes, driving oncogenic transformation or complex inflammatory processes. Myc has been called “the quintessential oncogene” and is deregulated in at least 40% of all human cancers, but the effects remain paradoxical. The broad transforming effect of c-Myc has been explained by its ability to bind to promoters of at least 30% of all known genes and in transgenic mice, c-Myc overexpression combined with inhibition of apoptosis is sufficient to drive pancreatic cancer formation. More recently, inhibition of c-Myc was proposed to stop cancer growth and even allow tissue repair and reversion to a functional phenotype. The study reported here suggests a naturally occurring mechanism of tissue-specific MYC expression, through upstream transcription factors. Defining the mechanism of tissue specific MYC regulation provides new ways to regulate c-MYC expression in a tissue specific manner. Furthermore, the molecular basis of bacterial MYC inhibition may be used to develop a new class of therapeutic MYC inhibitors with more tissue-specific functions. The suppression of MYC by treatment using a factor or bacteria as described above may provide a defense against oncogenic transformation, and thus treatment may be used prophylactically. In this case therefore, infection may be beneficial, unlike the stomach and H. pylori.

The invention will now be particularly described by way of example with reference to the accompanying diagrammatic drawings, the contents of which are as follows.

The patent or application contains at least one drawing executed in color. Copies of this patent or patent application publication with color drawing(s) will be provided by the Office upon request and payment of the necessary fee.

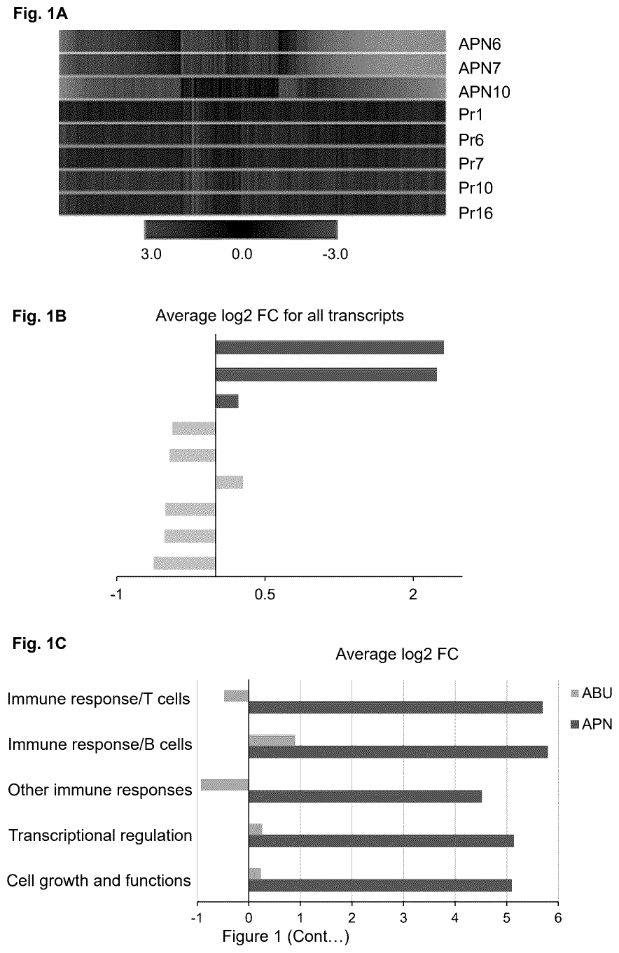

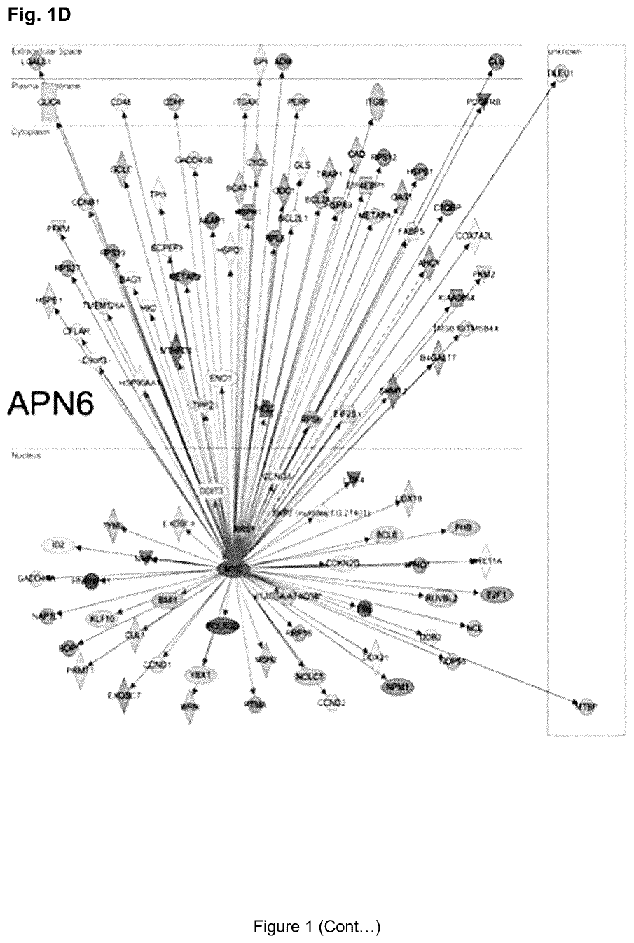

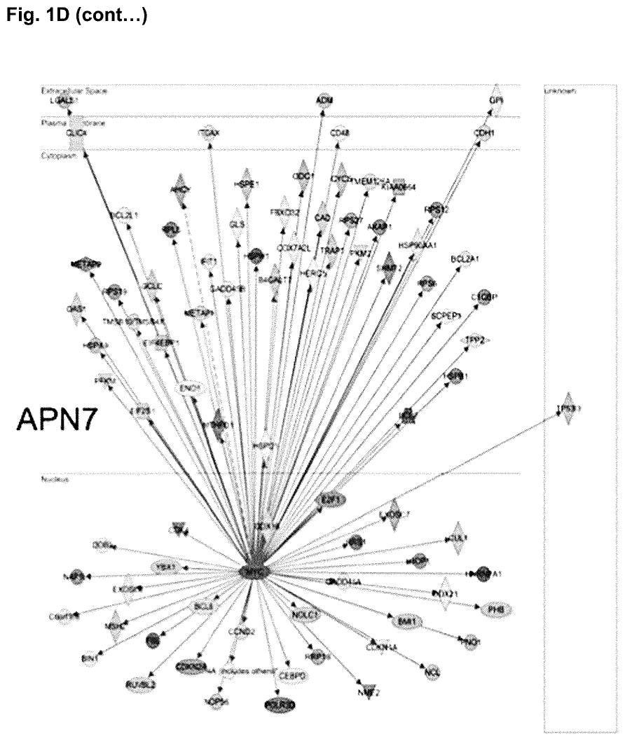

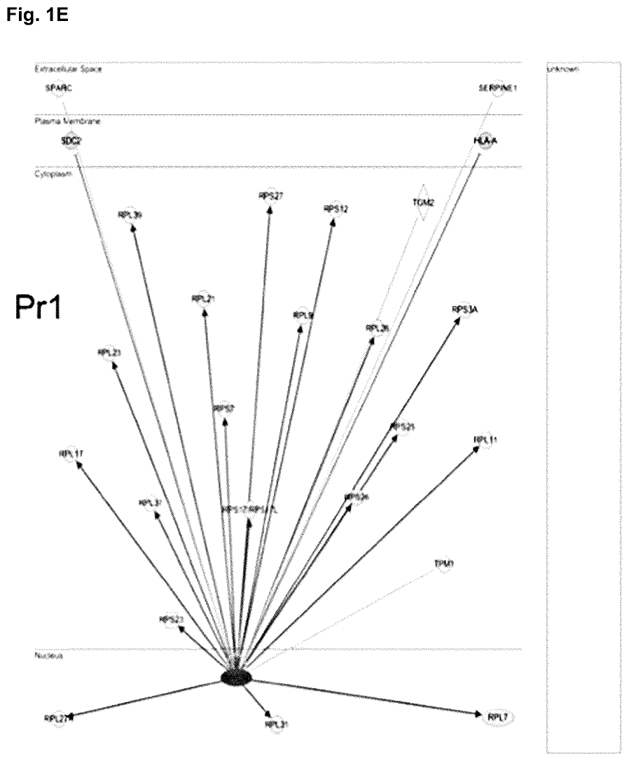

FIG. 1A-E. Regulation of MYC-related genes in children prone to acute pyelonephritis

(FIG. 1A) Heat map of 9118 RNA transcripts in patients with APN or ABU. The majority of genes were upregulated (dark grey) in the APN- but downregulated (light grey) in the ABU group. (FIG. 1B) Number of regulated genes in individual patients in the APN or ABU group. (FIG. 1C) Top canonical pathway analysis identified genes involved in adaptive immune responses and especially T-cell signaling. The corresponding genes were downregulated in the primary ABU group. (FIG. 1D) IPA network analysis of the MYC-related gene network in the APN patients. MYC, MYCN and KLF2 were activated in the APN group. (FIG. 1E) MYCN, JUN, TP53, HOXD3 were inhibited in the ABU patients group, and MYC related gene expression was inhibited.

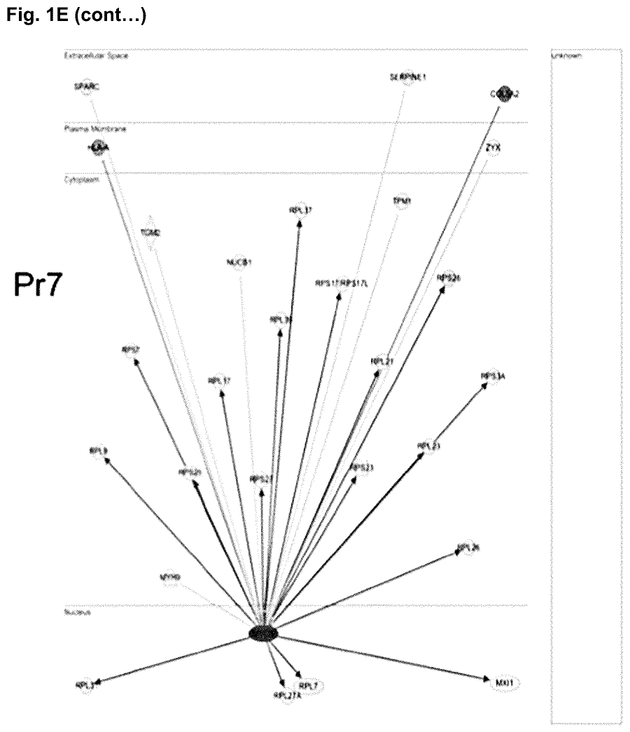

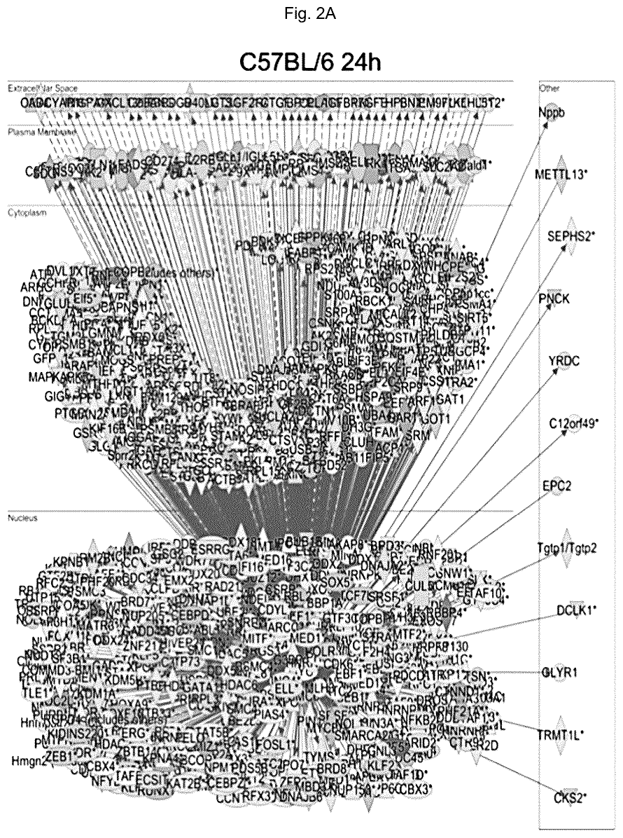

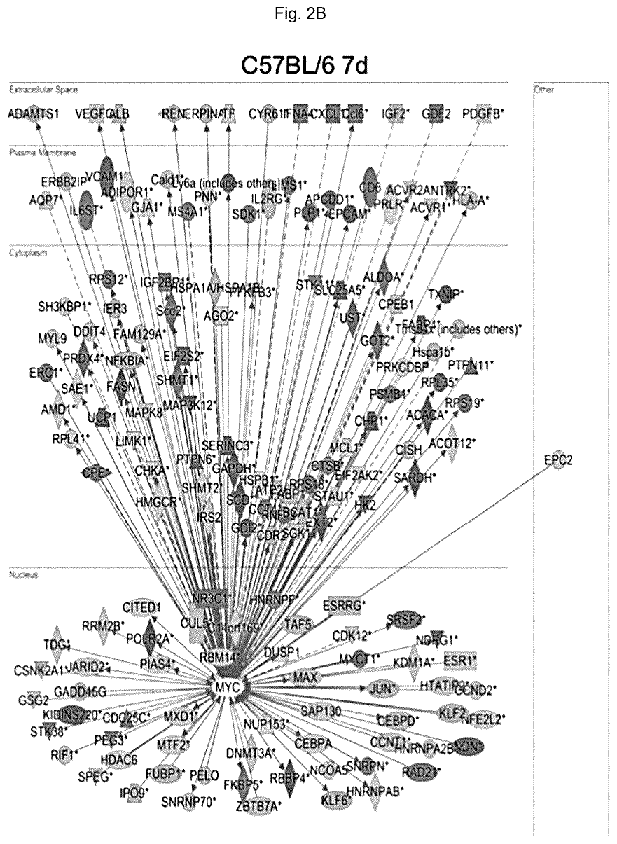

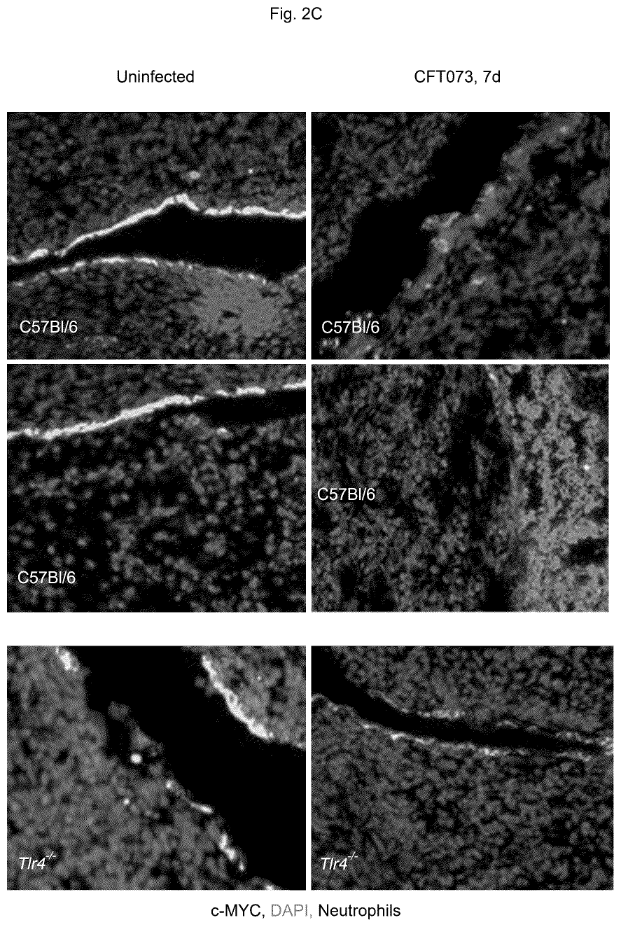

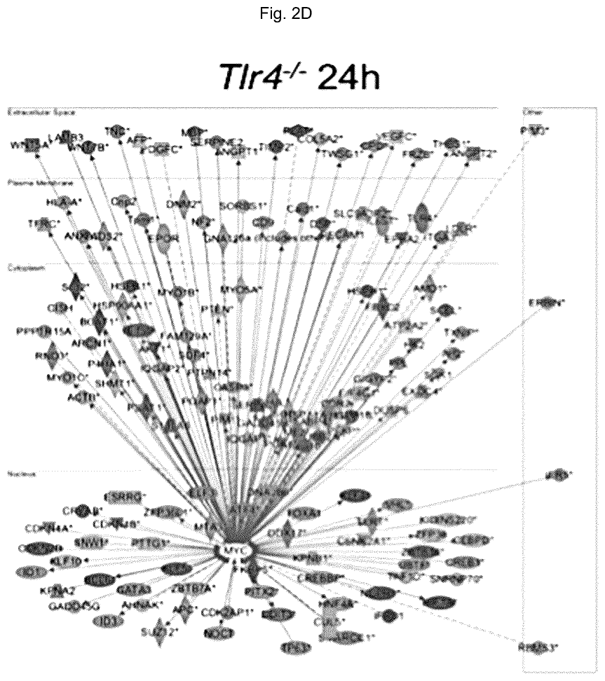

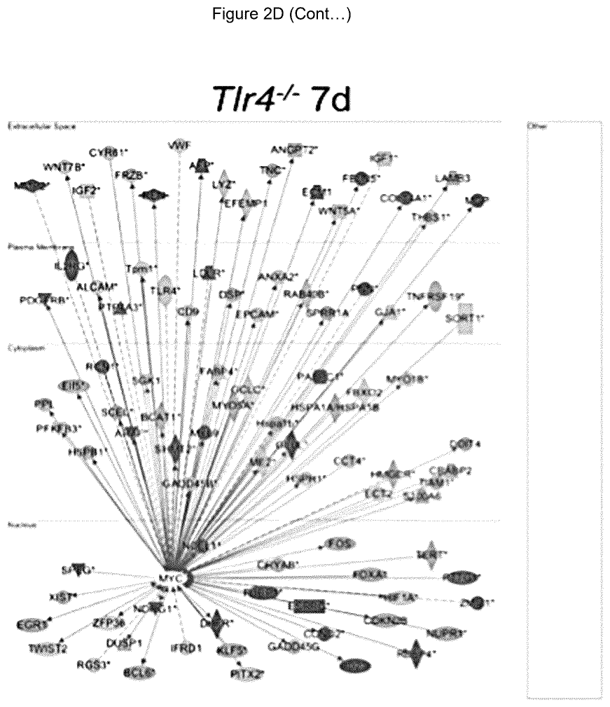

FIG. 2A-D. Inhibition of c-MYC expression in infected kidneys

(FIG. 2A) Massive down-regulation of MYC-related genes in C57BL/6 mice infected for 24 hours compared to (FIG. 2B) infected mice after 7 days. (FIG. 2C) Immunohistochemistry of frozen renal tissue sections showing the c-MYC expression in infected C57BL/6 mice (7 days) compared to uninfected C57BL/6 controls (top 4 panels).Degree of inflammation was evaluated in each mouse by neutrophils staining. Light grey, c-MYC; dark grey, neutrophils. Scale bar, 50 μm. Immunohistochemistry of frozen renal tissue sections showing epithelial c-MYC expression in Tlr4^{-/-} mice before and after infection (7 days) (bottom panels). Degree of inflammation was evaluated in each mouse by neutrophils staining. Light grey, c-MYC; mid grey, neutrophils; dark grey, nuclei. Scale bar, 50 μm. (FIG. 2D) Regulation of MYC-related genes in Tlr4^{-/-} mice infected for 24 hours and 7 days.

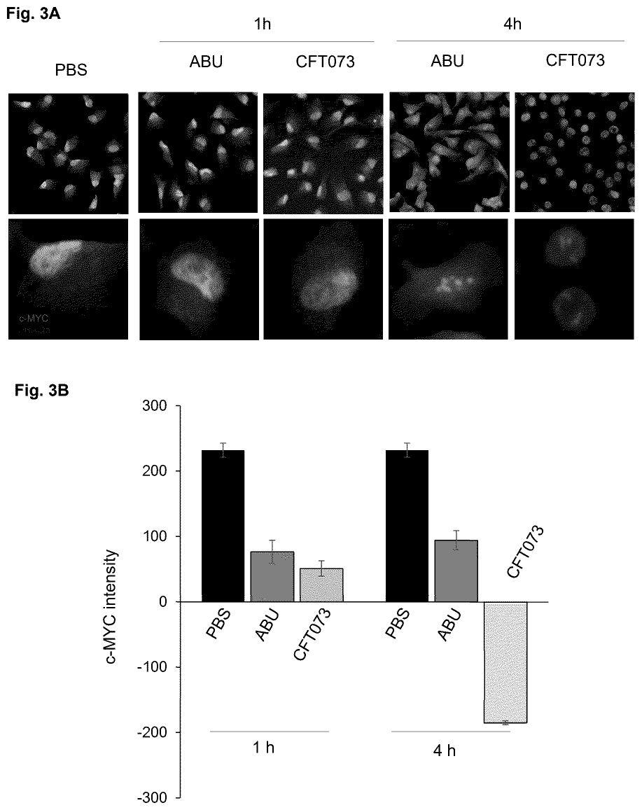

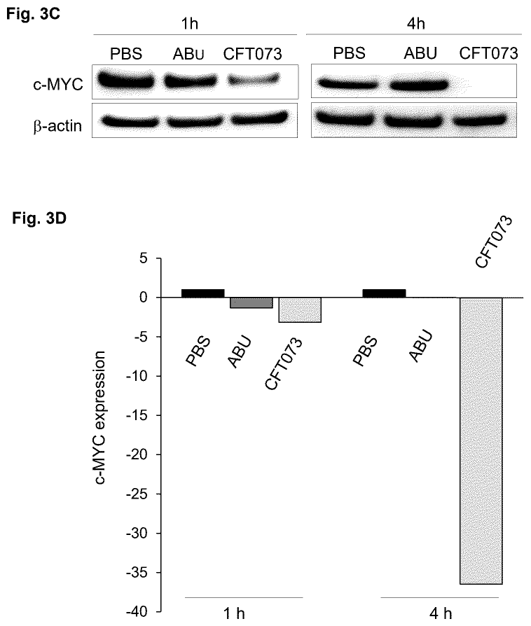

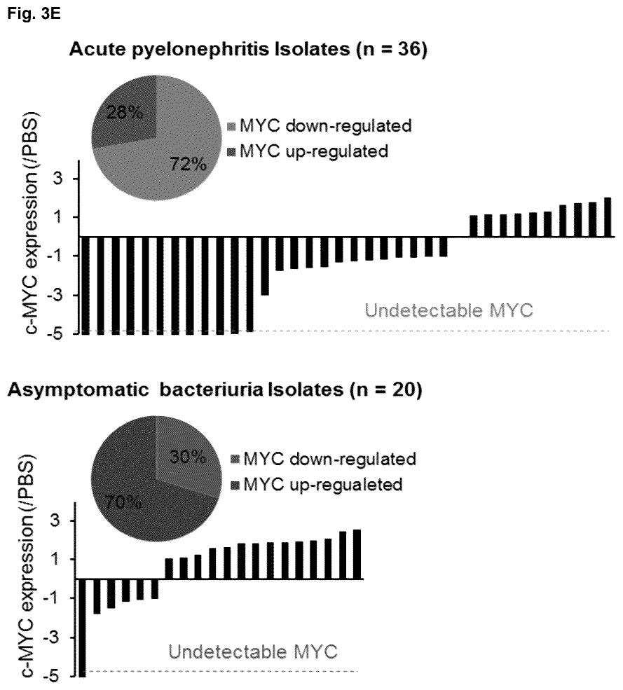

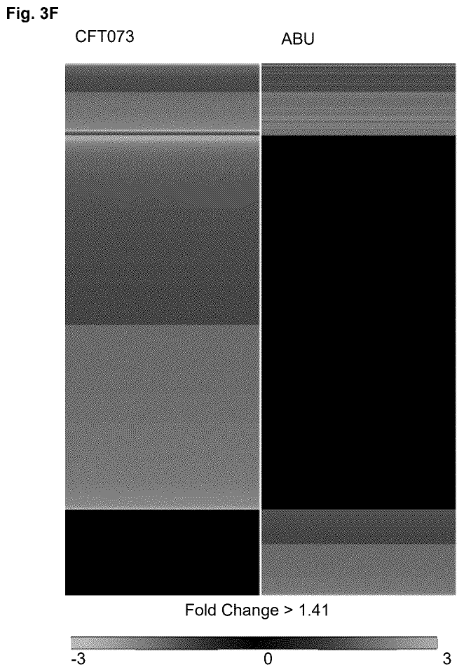

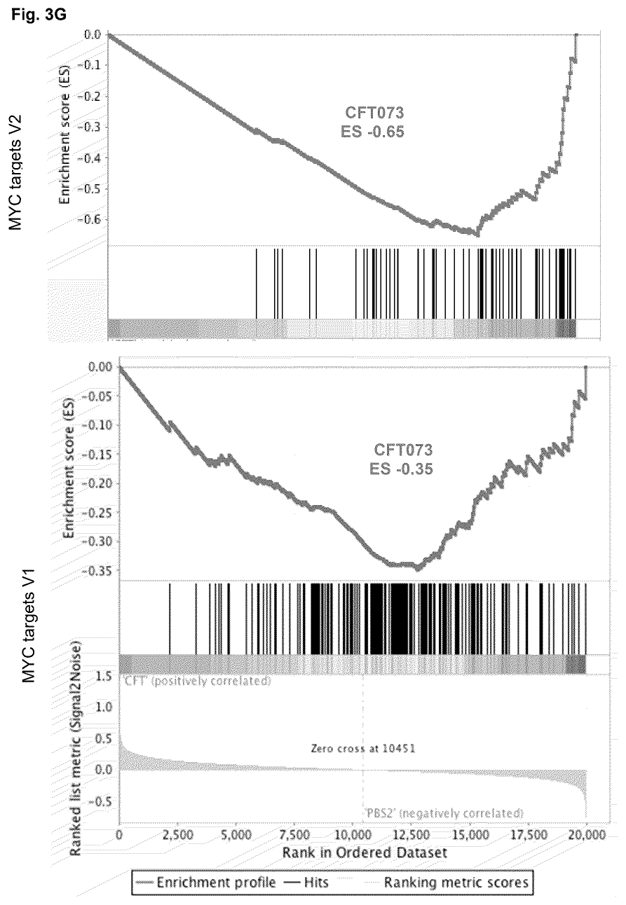

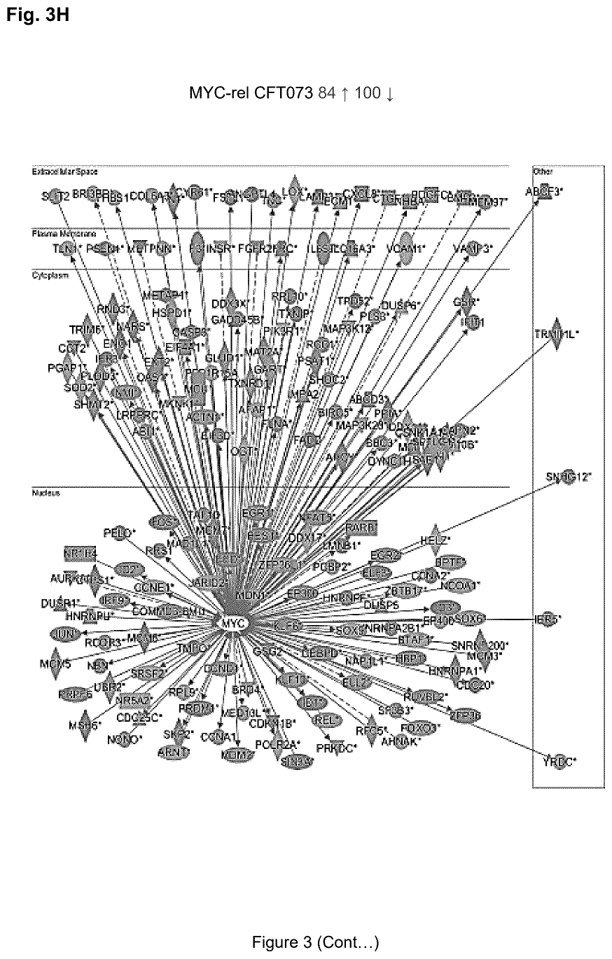

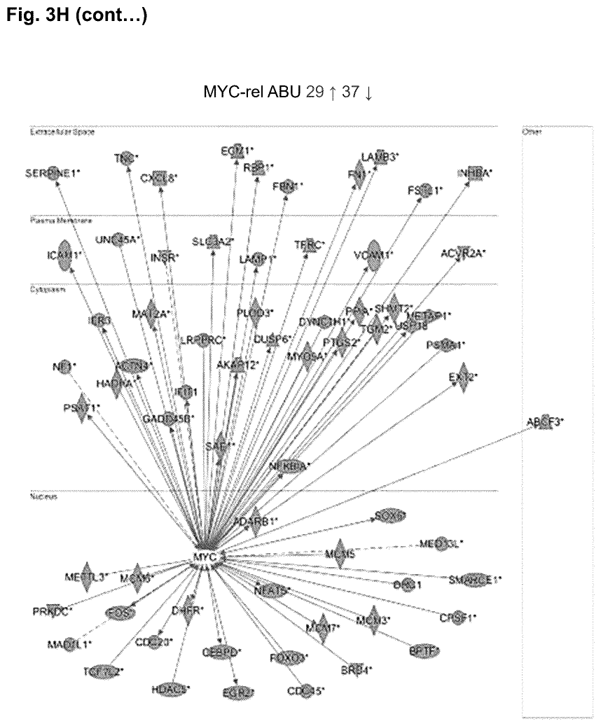

FIG. 3A-I. c-MYC suppression by acute pyelonephritis strains

(FIG. 3A) Effect of bacterial infection on epithelial c-MYC expression, quantified by confocal microscopy. A498 kidney epithelial cells were infected with CFT073 or the ABU strain E. coli 83972. CFT073 infection caused a decrease in c-MYC staining after 1 hour and a loss of staining after 4 hours. In contrast, MYC staining increased protein after infection with the ABU strain (1 and 4 hours) compared to uninfected control cells. Light grey=c-MYC; dark grey=nuclei. Scale bar 20 μm. (FIG. 3B) Quantification of staining in (FIG. 3A). (FIG. 3C) Western blot analysis showing decreased c-MYC staining in cells infected with CFT073 (1 and 4hours) and increased staining in cells infected with the ABU strain. (FIG. 3D) Quantification of the staining in (FIG. 3C). (FIG. 3E) Screening of clinical isolates for effects on c-MYC expression. A498 cells were infected for 4 hours) with acute pyelonephritis isolates (n=36) or asymptomatic bacteriuria isolates (n=20). c-MYC expression was quantified by Western blot analysis (one representative blot). (FIG. 3F) Heat map comparing the gene expression profiles of A498 cells infected with CFT073 or ABU (4 hours). Fold changes compared to uninfected controls (cutoff, 1.41). (FIG. 3G) Enrichment plot of MYC targets V2 (ES=−0.65) and V1 (ES=−0.35) in the CFT073 infected A498 cells (nominal P-value<0.05). GSEA—(FIG. 3H) IPA network analysis of the MYC-related gene network in A498 cells infected with CFT073 or ABU, as shown in FIG. 3F. (FIG. 3I) GSEA showing the top enriched gene sets in A498 cells infected with CFT073 (nominal P-value<0.05).

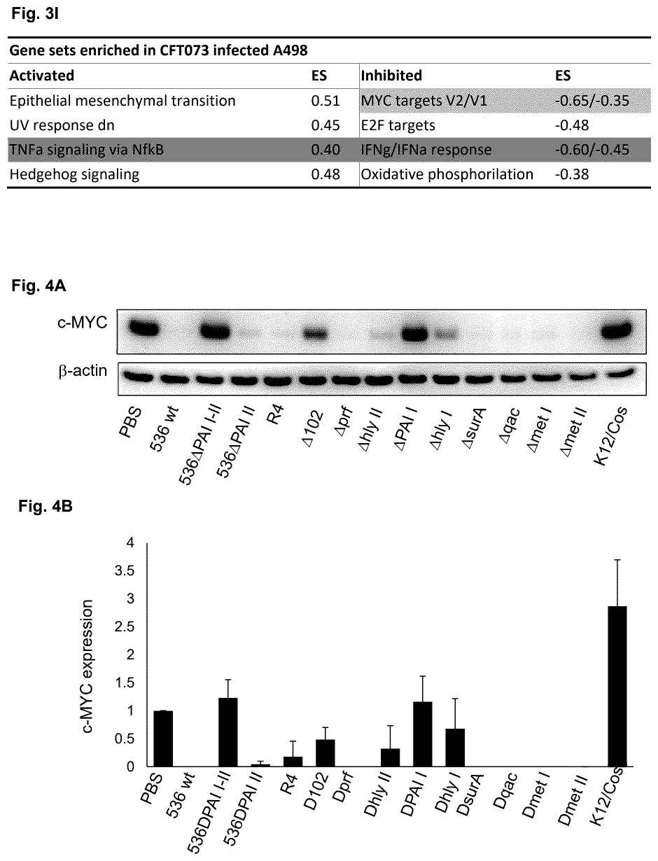

FIG. 4A-B. c-MYC suppression by 536 mutants lacking single genes located on PAI I or PAI II

(FIG. 4A) Identification of single genes involved in c-MYC suppression, using deletion mutants of genes coded on 536 PAI I or 536 PAI II. Western blot showing a complete c-MYC suppression after infection with E. coli 536 and c-MYC restore after infection with 536DPAI I, 536DPAI IDPAI II and 536 PAI II+/DleuX (536D102) and at less extend 536Dhly I, 536Dhly II, the staining in A498 cells infected with E. coli 536, which suppressed c-MYC expression, or mutants carrying deletions in PAI I-V and PAI I+II. MYC staining was regained in cells infected with the PAI I and PAI I+II deletion mutants, compared to the wild type strain. (FIG. 4B) Quantification of expression shown in FIG. 4A.

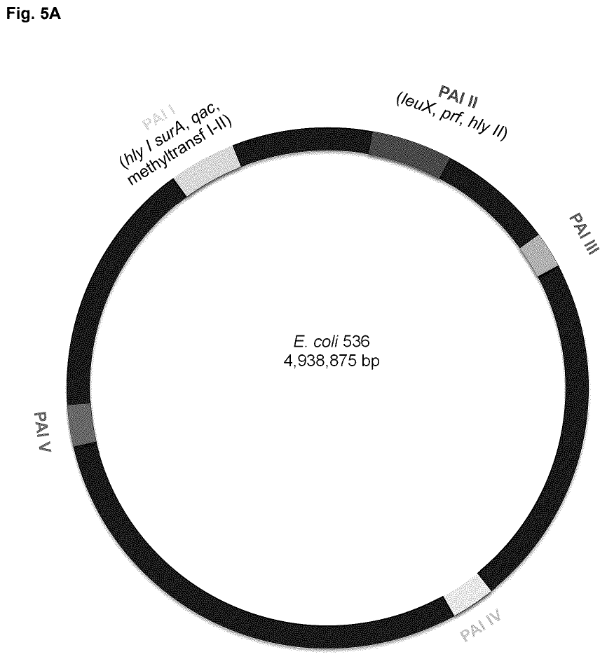

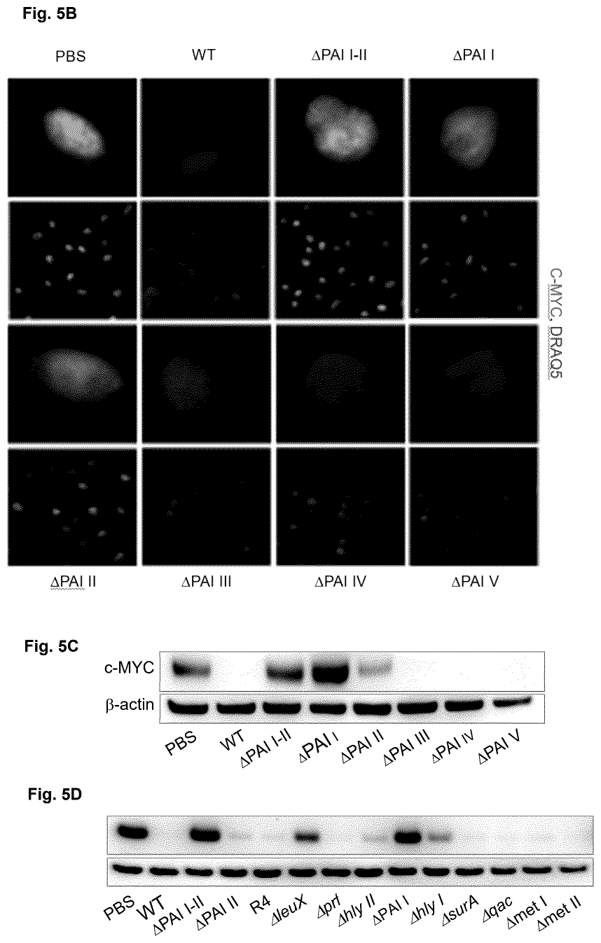

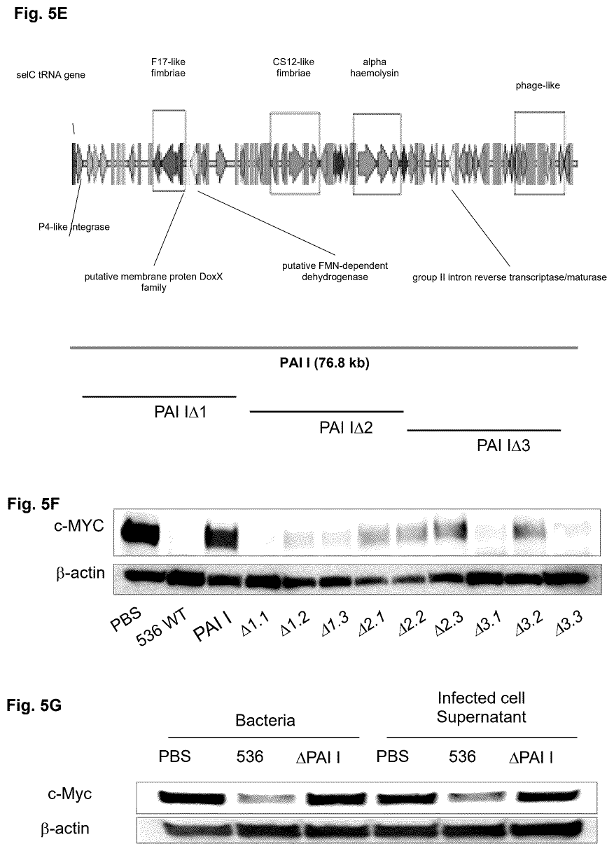

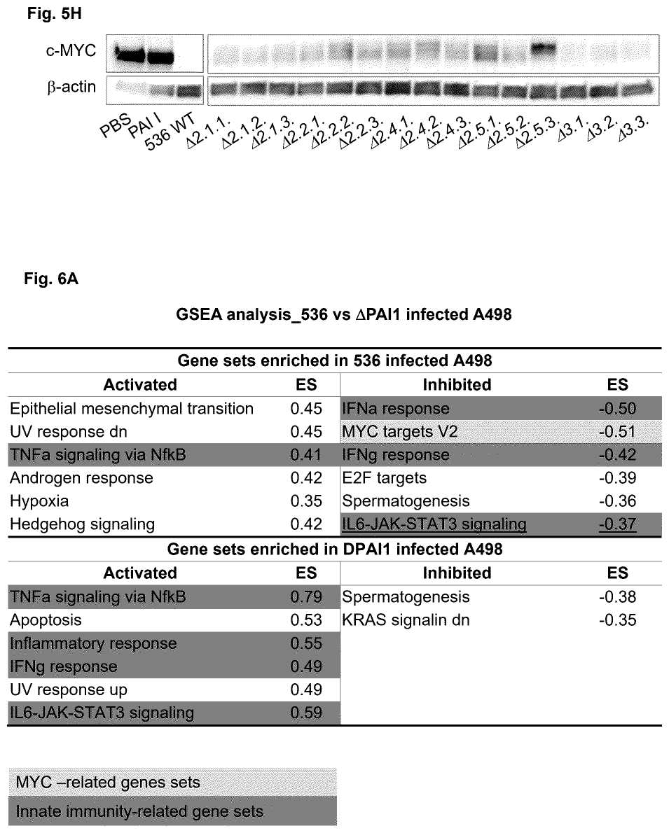

FIG. 5A-H Mechanism of bacterial c-MYC suppression

(FIG. 5A) Schematic representation of the E. coli 536 chromosome, indicating the positions of Pathogenicity Islands (PAIs) I-V. (FIG. 5B) Identification of chromosomal regions involved in c-MYC suppression, using deletion mutants. A498 cells were infected with E. coli 536 or mutants carrying deletions of PAI I-V or a PAI I+II double deletion (4 hours). c-MYC staining quantified by confocal microscopy (Light grey=c-MYC; dark grey=DRAQ5, nuclei). Scale bar 10 μm (one representative blot out of four). (FIG. 5C) Western blot analysis confirming the effects of the deletion mutants in FIG. 5B on c-MYC expression (left blot). (FIG. 5D) Western blot analysis of whole cell lysates in A498 cells infected with E. coli 536 mutants carrying single gene deletions on PAI I (ΔhlyI, ΔsurA, Δqac, Δmetyltranspherase I or Δmetyltransferase II) or PAI II (ΔPAI II/leuX+, PAI II+/ΔleuX and ΔhlyII). (FIG. 5E) Schematic representation of the sequential deletions of PAI I₅₃₆, in order to obtain the deletion mutants PAI IΔ1, PAI IΔ2 and PAI IΔ3. (FIG. 5F) Effect on c-MYC expression of sequential deletion of PAI I₅₃₆ analysed by Western blot (one representative blot out of two). Deletion mutants are labeled as Δ1.1, Δ1.2, Δ1.3, Δ2.1, Δ2.2, Δ2.3, Δ3.1, Δ3.2, and Δ3.3, and correspond to PAI IΔ1, PAI IΔ2 and PAI IΔ3, respectively. (FIG. 5G) Western blot analysis showing MYC degradation by supernatant from E. coli 536. (FIG. 5H) Western blot analysis of additional deletion mutants (Δ2.1.1, Δ2.1.2, Δ2.1.3, Δ2.2.1, Δ2.2.2, Δ2.2.3, Δ2.4.1, Δ2.4.2, Δ2.4.3, Δ2.5.1, Δ2.5.2, Δ2.5.3, Δ3.1, Δ3.2, and Δ3.3).

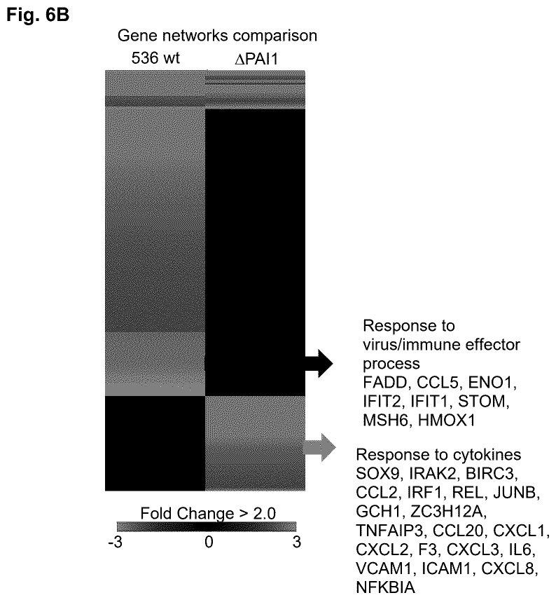

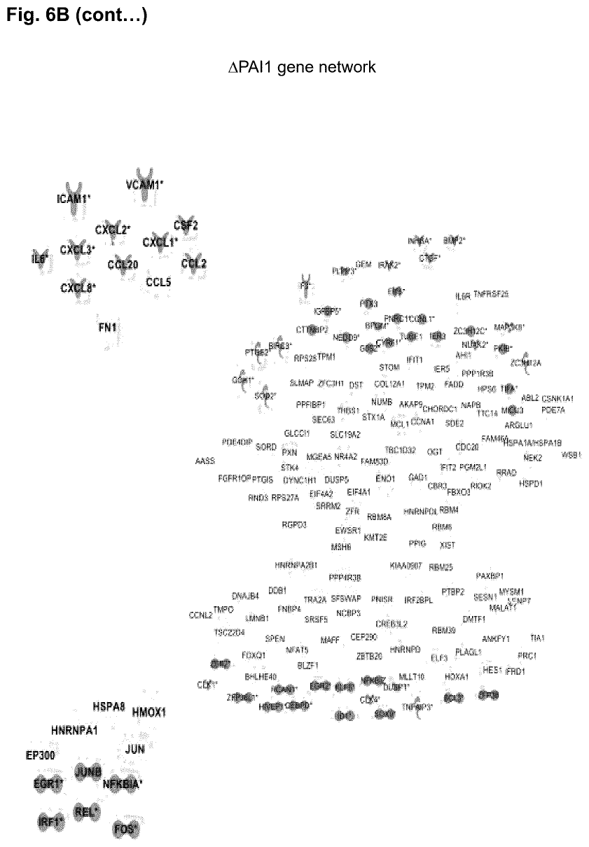

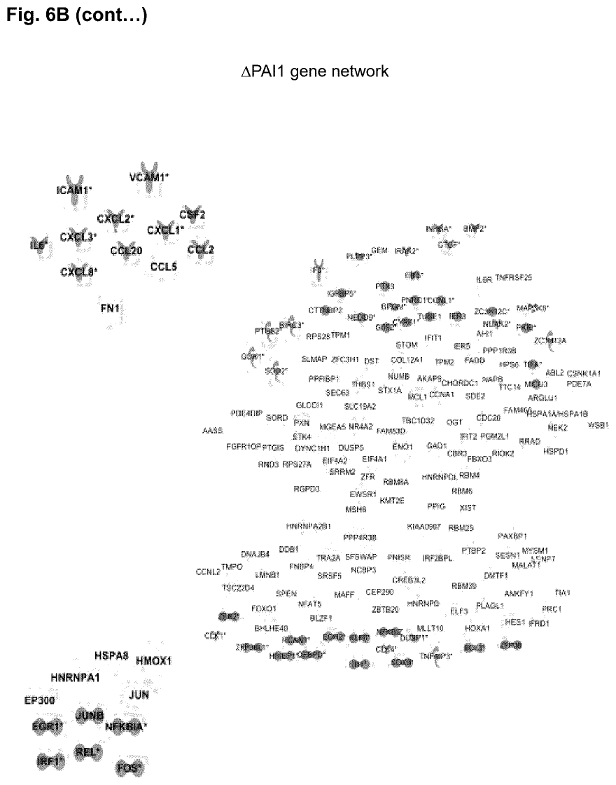

FIG. 6A-B Innate immune response down-regulation by E. coli 536 infection

(FIG. 6A) Heat map of genes regulated in 536 and 536ΔPAI I-infected cells and relative gene ontology analysis. The 536 infected cells showed down-regulation of different immune response genes. The same biological functions were activated by the 536ΔPAI I mutant. (FIG. 6B) IPA network analysis of the genes from the data set in (FIG. 6A). The molecular hubs were mainly chemokines, cytokines and transcription factors. The 536 WT strain did not activate an immune response, but the 536ΔPAI I mutant was a strong innate immune activator.

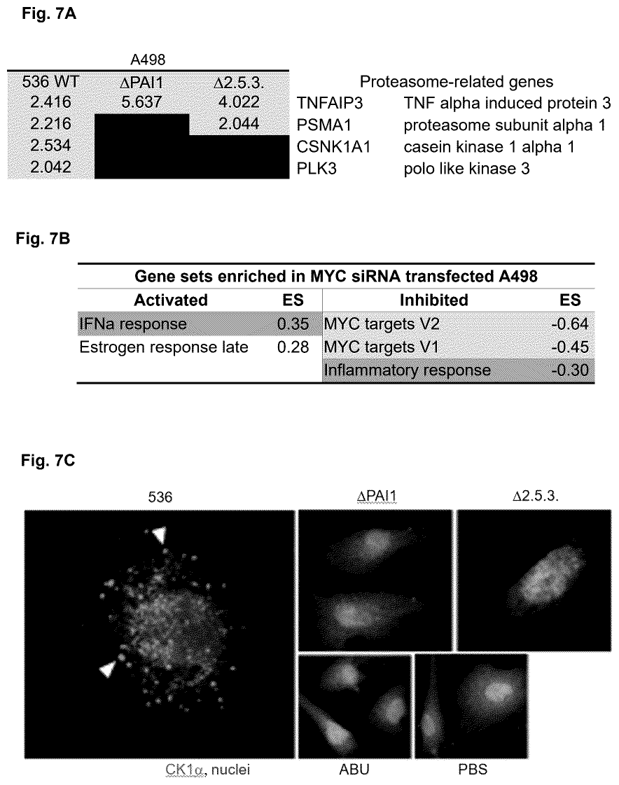

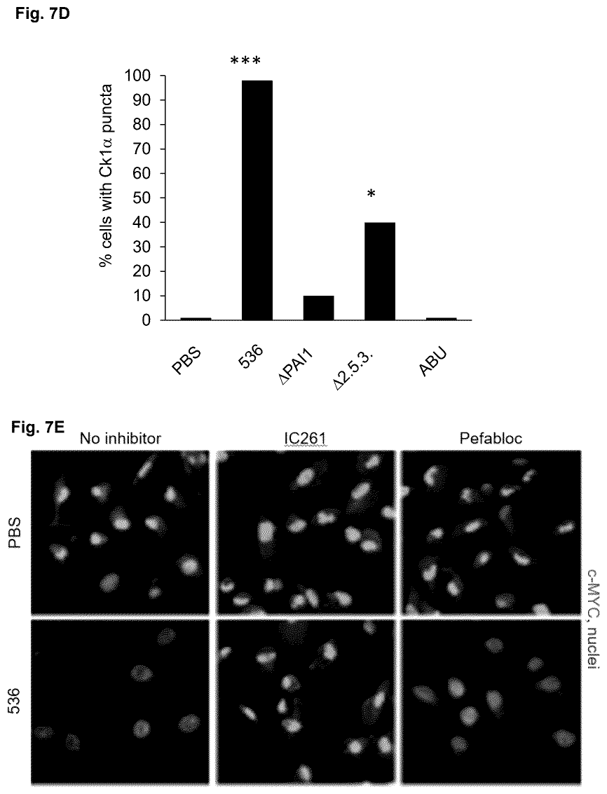

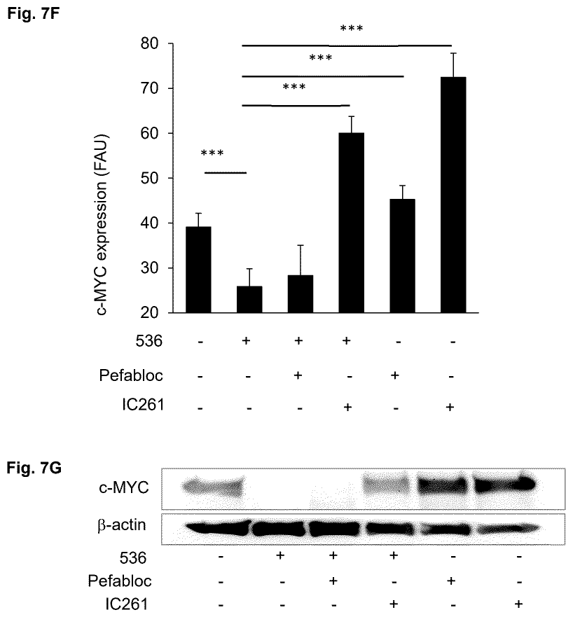

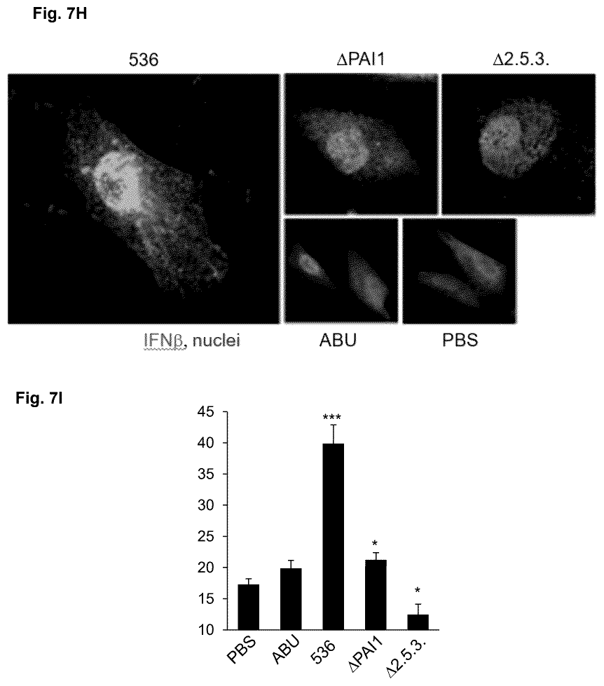

FIG. 7A-I c-MYC inhibition by Casein kinase 1 (CK1) and IFN activation.

(FIG. 7A) Heat map of proteasome-related genes regulated in 536, 536ΔPAI I and 536Δ2.5.3. infected cells. CSNK1A1 (CK1α1) was inversely regulated. Cut off fold change, 1.41 (FIG. 7B) Expression in MYC siRNA transfected cells. (FIG. 7C) CK1α expression in A498 cells infected with 536, 536ΔPAI I, 536Δ2.5.3. or ABU compared to uninfected PBS control. Confocal microscopy, Outer areas—CK1α; inner areas—nuclei. Scale bar 10 μm. (FIG. 7D) Activation of CK1α1 quantified as the number of cells showing punctate staining, characteristic of CK1α activation (***, P<0.001; *, P<0.5; χ² test). (FIG. 7E) The CK1 inhibitor (IC261) prevented c-MYC degradation. A498 cells treated with the Ck1 inhibitor (IC261) or the Serine proteases inhibitor (Pefabloc) for 30 minutes prior to infection. Confocal images of c-Myc expression in 536-infected cells. Scale bar 20 μm. (FIG. 7F) Quantification of the staining in FIG. 7E (***, P<0.001; t. test). (FIG. 7G) The results in FIG. 7E were confirmed by western blot analysis (one representative blot out of two). (FIG. 7H) IFNβ expression in A498 cells infected with 536, 536ΔPAI I, 536Δ2.5.3. and ABU and uninfected PBS control analyzed by confocal microscopy. Outer areas, IFNβ; inner areas—nuclei. Scale bar 10 μm. (FIG. 7I) Nuclear IFNβ staining in FIG. 7H (n.s.=non-significant;***, P<0.001; *, P<0.5; t. test).

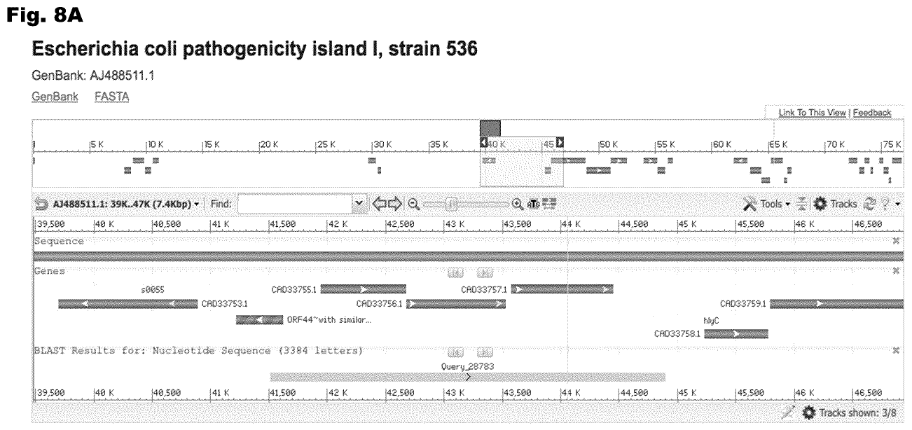

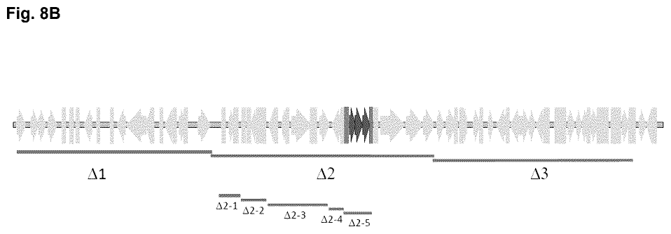

FIG. 8A is a gene map of Escherichia coli pathogenicity island I—strain 536 and FIG. 8B illustrates the partial deletions of PAI I of uropathogenic E. coli strain 536, showing ORFs deleted by deletion 2-5, where Δ2-1 represents ECP. 3821 (hypothetical protein), Δ2-2 represents ECP. 3822 (putative ABC metal-type transporter ATP-binding protein), Δ2-3 represents ECP. 3823 (putative ABC metal-type transporter permease), Δ2-4 represents ECP. 3824 (putative metal ABC transporter substrate-binding protein) and Δ2-5 represents ECP=0.3825 (hypothetical protein).

### Example 1

### C-MYC Regulates Transcription in UTI Prone Patients

The profiles of expressed genes in children with different UTI susceptibility profiles, defined by long-term clinical follow up, was assessed.

Children from southern Sweden with a history of UTI were enrolled in the study, after referral to one pediatric nephrologist. The patients were followed for at least six years at the Department of Pediatrics, Lund University Hospital and were assigned to three groups, depending on their UTI history. Children prone to APN had a history of acute pyelonephritis but no ABU episodes. Children assigned to the primary ABU group had no history of symptomatic UTI (APN or acute cystitis). Children who developed ABU after having completed antibacterial therapy for a prior symptomatic infection but with no further symptomatic episodes were assigned as secondary ABU.

A diagnosis of APN was based on fever (<38.5° C.) with significant bacteriuria, C-reactive protein (CRP) >20 mg/l and no symptoms of other infections. A diagnosis of ABU was based on at least three consecutive urine cultures yielding the same bacterial strain (>105 cfu/ml of urine) in a child with no symptoms of UTI and no CRP increase. while the other two The APN group had not developed ABU at any time during follow-up. However, the patients were infection-free at the time of sampling, and months and in some cases years had passed since the last UTI episode.

Pediatric controls were enrolled at the pediatric outpatient clinic or when admitted for elective surgery for diagnoses unrelated to infection. They had negative urine cultures at the time of sampling and no recorded history of UTI. Informed written consent was obtained from all participants or their parents/guardians. The study was approved by the Ethics Committee of the medical faculty, Lund University, Sweden (LU106-02, LU236-99).

RNA was purified from peripheral blood monocytes during an infection free interval and sequenced. Specifically, RNA was extracted using the QIAamp RNA blood Mini Kit (QIAGEN) or The PAXgene™ Blood RNA tube and PAX gene blood RNA system. After isolation, the RNA samples were stored at −80° C. until use. The quantity and quality of the RNA sample was evaluated using NanoDrop and bioanalyzer. The protocol from Illumina was used for making cDNA libraries. The patient's and control's total RNA was converted into a library of template molecules that are suitable for high throughput DNA sequencing. The poly-A containing mRNA molecules were purified using poly-T oligo-attached magnetic beads. Following purification, the mRNA was fragmented into small pieces using divalent cations under elevated temperature. Then the cleaved RNA fragments were copied into first strand cDNA using reverse transcriptase and random primers, followed by second strand CDNA synthesis using DNA Polymerase I and RNaseH. A single ‘A’ base was added to the cDNA fragments (an end repair process) and ligated to the adapters. These products were purified and enriched with PCR to create the final cDNA library.

The quality of the library was evaluated using the Agilent Technologies 2100 Bioanalyzer and a DNA 1000-kit. The quantity of adapter ligated fragments was determined by qPCR using the KAPA SYBR FAST library quantification kit for Illumina GA (KAPA Biosystems). A 10 pM solution of the sequencing library was used in the cluster generation on the Cluster Station (Illumina Inc.). Paired-end sequencing with 100 bases read length was performed using the Genome Analyzer_{IIx}, according to the manufacturer's protocols. Images were base called and and quality filtered using the analysis pipe-line supplied together with the Genome Analyzer instrument. The human genome reference sequence was Hg37.

Sequence reads were mapped using Tophat using default settings and reads were aligned to human reference genome 37.2. Cufflinks was then used to process the Tophat alignments and assemble reads into transcripts. The abundance of the assembled transcripts was estimated as Fragments Per Kilobase of exon per Million fragments mapped (FPKM). The assembled Cufflinks transcripts were then processed and patient samples log₂-normalized to controls before bioinformatics analysis.

Ingenuity Pathways Analysis (IPA) (Ingenuity Systems) was used to relate changes in gene expression to functional changes and to discover canonical pathway activation and the biological interaction networks among regulated genes. The input gene lists included genes with a log2 fold-change cut-off of 0.8. The transcripts obtained by RNA sequencing were quantified compared to age matched controls with no history of UTI. The overall transcriptional activity was markedly higher in the acute pyelonephritis (APN) prone group than in children prone to asymptomatic bacteriuria (ABU), as shown by the number of transcribed genes and the level of individual gene transcripts (p<0.05, n=10154, 9854 or 8030 in APN6, APN7 or APN10 and n=1779, 2577, 2955, 5356, 3213 in Pr1, Pr6, Pr7, Pr10 and Pr16, respectively, Mean+range with ±0.8 log2 fold-change over the pediatric controls), (FIG. 1A) and by an average gene expression of 1.6 log2 fold change in the APN group compared to −0.4 log2 fold change for the ABU group (FIG. 1B).

Distinct transcriptional profiles distinguished the APN from the ABU group. Genes involved in T-cell function and signaling were strongly upregulated in the APN group (FIG. 1C) but were suppressed in ABU (FIG. 1C), as confirmed by GSEA (FIG. 3SB, NES=−1.7552, nominal p-value=0.0147). In the ABU group, Type 1 Interferon genes predominated, consistent with IRF3, IRF7 and IRF1 activation (Table 1). Also, most of the STATs, SOCS 1, IFIT1 and IFIT3, OAS1, MX1 and IFI35 were upregulated, indicating transcriptional activity downstream of IRF7.

Transcriptional regulator analysis predicted that MYC, MYCN and KLF2 were activated in the APN group (FIG. 1D and Table 2 MYCs), based on the expression of known targets and validation by two complementary statistical tests, generating a p-value and a regulation z-score. MYC, PAX5 and MAF were up-regulated in the APN group and about 66% of downstream genes had an expression pattern consistent with MYC activation (Table 2). Regulation of the MYC transcription factors was confirmed by Gene Set Enrichment Analysis (GSEA) analysis.

GSEA was used to assess whether a defined set of genes showed statistically significant, concordant differences between the patient groups. The log2 fold-change calculated from the FPKM values from patients and controls were calculated. The genes were then ranked by the order of expression in the patient group. This list was uploaded as a pre-ranked gene list to GSEA v2.04 (Broad Institute, Cambridge, MA) and GSEA with emphases on canonical pathways and transcription factors used.

The MYCMAX transcription factor motif was strongly enriched in the APN group as was confirmed from (GSEA, where the MYC/MAX transcriptional motif was the most highly enriched. In contrast, c-MYC expression was not regulated in the ABU prone group and the MYC-related genes were predicted to be globally down-regulated (FIG. 1E). This was also confirmed by GSEA analysis.

The results suggest that c-MYC is upregulated in APN prone patients, during infection free intervals, resulting in a transcriptionally hyperactive phenotype that includes different T cell functions but not innate immunity.

### Example 2

### Uropathogenic E. coli Inhibit c-MYC Expression in the Renal Pelvic Mucosa

To study if infection modified c-MYC expression, acute pyelonpehritis infections in C57BL/6 wild type mice were established and the profile of regulated genes characterised. Mice were infected by intra-vesical inoculation with the uropathogenic E. coli strain CFT073 (an overnight static cultures of E. coli CFT073 in Luria broth) and RNA for genome-wide transcriptomic analysis was obtained at sacrifice after 24 hours or seven days.

a

Specifically, mice were housed in specific pathogen-free individually ventilated cages (IVC) at a constant temperature of 23° C. on a 12-h light-dark cycle with lights on at 7:00 a.m, with food and water ad libitum. Female C57BL/6 wild type, T1r4^{-/-}, Irf3^{-/-} and Irf7^{-/-} and mice were used at 9-15 weeks of age. Irf7^{-/-} and Irf3^{-/-} mice on a C57BL/6 background were kindly provided by the Riken Bioresource Center, Japan, with permission from T. Taniguchi.

Mice anesthetized with Isofluoran were infected by intra-vesical inoculation with E. coli CFT073 (10⁸ CFU in 0.1 mL) (28), and sacrificed under anesthesia. Kidneys were aseptically removed and macroscopic pathology was documented by photography. One kidney was divided for RNA extraction (on dry ice) and immunostaining (in O.C.T. compound, VWR).

Macroscopic pathology was documented by photography and frozen tissue sections were processed for H&E staining and immunohistochemistry was used to assess MYC expression and the degree of inflammation and tissue damage.

Tissues embedded and frozen in O.C.T. were cryosectioned (8 μm, Leica microtome), collected on positively charged microscope slides (Superfrost/Plus; Thermo Fisher Scientific), fixed in acetone-methanol (1:1, 10 min), dried, permeabilized (0.2% Triton X-100, 5% normal goat serum/PBS) and stained with primary rat anti-neutrophil-antibody [NIMP-R14] (1:200; Abcam, ab2557), rabbit monoclonal anti-c-Myc antibody (1:100; ab32072, Abcam), followed by Alexa 488 or Alexa 568 labeled rabbit-anti-rat and goat-anti-rabbit IgG secondary antibodies (Cell Signaling). Nuclei were counterstained with DAPI (0.05 mM; Sigma-Aldrich). Slides were examined by fluorescence microscopy (AX60, Olympus Optical).

Urine samples were analysed for the presence of bacteria and neutrophils.

C57BL/6wt mice showed intact kidneys, rapid neutrophils infiltration and moderate bacterial counts in the renal pelvis and in urine after 24 hours. A moderate disease response was also documented after 7 days, with rapid neutrophil activation and a gradual clearance of infection.

Surprisingly, gene expression analysis revealed a massive inhibitory effect on the expression of MYC-related genes, 24 hours after infection (808 down-regulated and 190 up-regulated genes, FIG. 2A). After seven days, this response had subsided but a smaller number of MYC-related genes were regulated, compared to uninfected mice (66 up- and 96 down-regulated), (FIG. 2B).

The reduction in c-MYC expression was confirmed by immunohistochemistry, using specific antibodies. Kidney sections from uninfected mice, revealed an esclusively mucosal c-MYC staining pattern, suggestive of epithelial expression (FIG. 2C). c-MYC staining was virtually undetectable after infection.

### Example 3

### c-MYC Regulation by the Irf3 and Irf7 Transcription Factors

The Irf3 and Irf7 transcription factors have recently been identified as key susceptibility determinants in acute pyelonephritis. Specifically, Irf7 was shown to drive the development of pathology in Irf3^{-/-} mice (Puthia et al. 2016). Irf3^{-/-} mice developed severe pathology, with absescess and kidney edema, neutrophil infiltration into the renal papilla and pelvic mucosa. Neutrophils counts in urine were elevated after 24 hours and 7 days and bacteria invaded into the renal pelvis, papillae and collecting ducts. On the contrary, Irf7^{-/-} mice showed macroscopically unaffected kidneys and bacterial and neutrophil counts were even lower than in wild-type mice. Neutrophils counts in urine reached a transcient peak after 24 hours, but decreased rapidly.

By comparing Irf3^{-/-} to Irf7^{-/-} mice, we observed potent, divergent effects on MYC expression. After initial inhibition of c-MYC and c-MYC-dependent genes in both genotypes MYC was activated in Irf3^{-/-} mice, as were MYC-related genes (311 up-regulated and 91 down-regulated). This effect was not observed in Irf7^{-/-} mice, where fewer genes were regulated and the majority was suppressed (46 up- and 84 down-regulated).

During the early stage of infection, MYC was inhibited and c-MYC dependent genes showed an inhibitory profile. More than 1000 genes were regulated and regulated genes were mostly suppressed. In Irf3^{-/-}, mice, where infection remained, there was a transition from inhibition to activation of about 300 genes. This transition did not occur in Irf7^{-/-}, mice, suggesting that the MYC response in regulated by Irf7 in the Irf3^{-/-} background. This is consistent with the known kinetics of Irf7 expression, which requires activation and therefore is expressed during the later stage of infection.

MYC was expressed in the renal pelvic epithelium of uninfected Irf3^{-/-} mice but staining was lost after infection (data not shown). The staining pattern of uninfected and infected Irf3^{-/-} mice resembled that in C57BL/6 mice (data not shown). The Irf7^{-/-} mice, in contrast, showed weak epithelial MYC staining prior to infection and no further change after infection (data not shown).

The results suggest that the epithelial C-MYC response is Irf3 and Irf7 dependent, with a massive suppression after 24 hours followed by upregulation of MYC when IRF7 is expressed, in mice lacking Irf3. This increase in MYC expression and related genes infection did not occur in the absence of Irf7.

### Example 4

### Specificity for Irf7, Defined by siRNA Inhibition Therapy

To address if the effect on c-MYC expression is Irf7-specific, d liposomal Irf7 siRNA was administered to Irf3^{-/-} mice.

Irf3^{-/-} mice were treated with a siRNA dose of 5 mg/kg. A dose of 300 μL of Silencer Select Pre-designed Irf7 siRNA (Life Technologies, 4404010 #s79411) was injected into the tail vein (200 μL) and intravesically (100 μL) using Invivofectamine reagent (Life Technologies, 1377501). Irf7 siRNA was injected three days prior to infection and on the day of infection with E. coli CFT073 for a preventive Irf7 reduction. For therapeutic purpose, mice were treated with the Irf7 siRNA 24 hours and three days after infection. Ambion In Vivo Negative Control siRNA was used as a negative control (Life Technologies, 4457287).

### Transcriptomics Analysis

Total RNA was isolated from mice kidneys using mirVana miRNA Isolation Kit (Ambion by Life Technologies) followed by organic extraction using Acid-Phenol:Chloroform. Mouse Genome 430 PM array strips in a GeneAtlas (Affymetrix) was used for the analysis.

Irf7-specific siRNA was compared to scrambled, control RNA and in addition, untreated mice were used as controls. Irf7 siRNA treatment has previously been shown to inhibit epithelial IRF7 expression in infected kidneys and to protect mice from inflammation and tissue damage associated with acute pyelonephrtitis.

The transcription of Myc-related genes was drastically changed after Irf7 siRNA treatment of Irf3^{-/-} mice. 120 MYC-related genes were regulated exclusively in the Irf3^{-/-} mice (77 up- and 43 down-regulated). Treatment reversed the increase in MYC activity during the later stage of infection and MYC related gene networks were less stongly activated. In addition, 19 genes, were specifically regulated in the treated mice.

Importantly, treatment with Irf7 specific siRNA prevented the regulation of all these genes, suggesting that MYC acts downstream of the IRFs.

### Example 5

### Bacteria Suppress c-MYC Expression in Infected Human Kidney Cells

To further understand the molecular basis of the c-MYC response, we infected human kidney epithelial cells with the pathogenic strains CFT073 and 536 or with the ABU strain E. coli 83972.

The ABU strain E. coli 83972 (OR:K5:H-) (27, 48-50), prototype APN strains E. coli CFT073 (06:K2:H1) (47), 536 (06:K15:H31) wild type as well as the 536ΔPAI I (536-114), 536ΔPAI II (536-225), 536ΔPAI III, 536ΔPAI IV, 536ΔPAI V, 536ΔPAI IΔPAI II (536-21), 536 PAI II′/ΔleuX (536D102), 536ΔPAI II/LeuX+ (536R4), 536 ΔMethyltransferase I, 536 ΔMethyltransferase II, 536Δqac, 536Δhly I, 536Δhly II, 536Δprf and 536ΔsurA were cultured on tryptic soy agar (TSA) plates (16 hours, 37° C.), harvested in phosphate-buffered saline (PBS, pH 7.2, 10¹⁰ CFU/mL) and diluted as appropriate.

The A498 human kidney epithelial cells (A498, ATCC HTB-44) were cultured in RPMI-1640 supplemented with 1 mM sodium pyruvate, 1 mM non-essential amino acids and 10% fetal bovine serum (FBS) overnight at 37° C., 90% humidity and 5% CO₂ in 6-well plates (for western blots), or 8-well chamber slides (for confocal imaging). Cells were then infected with bacteria in fresh, serum-free supplemented RPMI.

After 1 and 4 hours, c-MYC expression was quantified by confocal microscopy and western blots. For confocal microscopy, A498 cells were infected with ABU and CFT073 for 4 hours, fixed (3.7% formaldehyde, 10 min), permeabilized (0.25% Triton X-100, 5% FBS, 15 min), blocked (5% FBS, 1 h at RT), incubated with anti-c-Myc primary antibody (1:1000; ab32072, Abcam) in 5% FBS overnight at 4° C. and Alexa Fluor® 488 goat anti-rabbit IgG secondary antibody (1:200; A-11034, Life Technologies) for 1 h at RT. After nuclear staining (DRAQ5, Abcam), slides were mounted (Fluoromount, Sigma-Aldrich), imaged by laser-scanning confocal microscopy (LSM510 META confocal microscope, Carl Zeiss) and quantified by ImageJ software 1.46r (NIH).

For Western blotting, cells were cultured in 6 well plates (4×10⁵ cells/well, Thermo Fisher Scientific), washed with ice-cold PBS and lysed with RIPA buffer containing protease and phosphatase inhibitors (Roche Diagnostics). Cell lysates were run on SDS-PAGE (4-12% Bis-Tris gels, Invitrogen) and blotted onto PVDF membrane (GE Healthcare), blocked with 5% bovine serine albumin (BSA) and incubated with rabbit anti-c-Myc primary antibody (1:1000 in 5% BSA). The blots were washed with PBS tween 0.1% (PBST) and incubated with secondary antibodies in 5% non-fat dry milk (NFDM) (1:4000, goat anti rabbit-HRP, Cell Signaling), then washed again with PBST, developed with ECL Plus detection reagent (GE Health Care) and imaged using the Bio-RAD ChemiDoc™ system. β-actin (1:4,000, A5441, Sigma) was used as loading control.

The results are shown in FIGS. 3A-D. Consistent with the in vivo data, CFT073 caused a massive decrease in c-MYC staining intensity. In contrast, the effect of the ABU strain was less pronounced. These effects were confirmed by Westen blot analysis.

The asymptomatic bacteriuria (ABU) strain E. coli 83972, in contrast, did not reduce c-MYC levels, suggesting that the ability to inhibit MYC might be virulence related (FIG. 3A-D).

This hypothesis was tested by screening a collection of clinically defined APN (n=36) and ABU (n=20) strains for effects on c-MYC expression in the human kidney cells (FIG. 3E-F). Compared to uninfected cells, MYC protein levels were reduced by 72% of the APN strains and 30% of the ABU strains.

The effects of infection on host gene expression were compared in human kidney cells infected with the CFT073 or ABU strains (FIG. 3G). In CFT073 infected cells, the top scoring gene sets included MYC related genes, with inhibition indicated by negative enrichment scores (−0.65 and −0.35, FIG. 3H).

Positively enriched genes were involved in epithelial-mesenchymal transition and NFkB dependent IFN and TNF responses. Negatively regulated gene sets included IFNs and E2F targets. The ABU, strain, in contrast, did not significantly regulate MYC-dependent genes. This difference between CFT073 and ABU was further illustrated by analysis of MYC-dependent gene networks (FIG. 3I).

The results show that uropathogenic E. coli inhibit mucosal c-MYC expression in vitro and that MYC-dependent gene networks are attenuated.

The specificity of these bacterial effects for MYC was supported by siRNA transfection of the human kidney cells. By gene expression analysis and GSEA, we identified the same gene sets as being suppressed by the siRNA and CFT073.

To understand whether c-MYC down-regulation by the UPEC strains is linked to specific virulence factors, the E. coli 536 wild type strain and mutants carring deletions in specific chromosomal regions (536ΔPAI I (536-114), 535ΔPAI II (536-225), 536ΔPAI III, 536ΔPAI IV, 536ΔPAI V and 536ΔPAI IΔPAI II (536-21) were further examined. The 536ΔPAI I (536-114) and 536ΔPAI II (536-225) mutants had lost the ability to inhibit c-MYC expression, suggesting that genes encoded on PAI I and/or PAI II are responsible for inhibiting the c-MYC response. In contrast, the 536ΔPAI III, 536ΔPAI IV and 536ΔPAI V mutant strains were phenotypically similar to E. coli 536 wild type. Cells infected with the 536ΔPAI IΔPAI II (536-21) strain, lacking both the PAIs I and II, showed the same c-MYC activation as the single PAI I mutant.

To identify specific genes responsible for the suppression of c-MYC, human kidney epithelial cells were infected with mutants carrying deletions in PAI II (ΔPAI II/leuX+, PAI II+/ΔleuX and ΔhlyII) and five mutants carrying deletions in PAI I (ΔhlyI, ΔsurA, Δqac, Δmetyltranspherase I and Δmetyltransferase II). By western blot analysis, the PAI I, leuX, hly II and hly I mutants showed an increase in c-MYC staining compared to the wild type strain (FIG. 4A).

The results suggest that PAI I is involved in the suppression of epithelial c-MYC expression, with contributions of leuX on PAI II and hly, on PAI I and II.

### Example 6

### Further Identification of the Bacterial c-MYC Inhibitor

As discussed above, the mechanism of bacterial c-MYC inhibition was defined, by genetic analysis of the uropathogenic strain E. coli 536 in which pathogenicity islands (PAIs) and virulence genes have been extensively characterized (FIG. 5A). E. coli 536 caused a virtually complete loss of c-MYC staining in human kidney cells after four hours, reproducing the effect of E. coli CFT 073 (FIG. 5B). A set of mutants with defined chromosomal deletions were constructed and evaluated for effects of epithelial MYC expression, by infection of the human kidney epithelial cells.

In this case, the bacterial MYC inhibitor was localized to PAI I (FIG. 5A-C). E. coli 536ΔPAI I (536-114) was identified as a loss of MYC inhibition mutant and the 536ΔPAI/IΔPAI II (536-21) double deletion mutant showed the same loss of function phenotype as 536ΔPAI I. The 536ΔPAI II (536-225), 536ΔPAI III, 536ΔPAI IV and 536ΔPAI V deletion mutants, in contrast, maintained the inhibitory phenotype of the WT strain (FIG. 5B-C) as did mutant strains carrying specific virulence gene deletions within PAI I (ΔhlyI, ΔsurA, Δqac, Δmetyltranspherase I and Δmetyltransferase II), (FIG. 5C). A PAI II+/ΔleuX mutant showed a loss of MYC inhibitory activity.

Partial PAI I deletion mutants were subsequently generated using lambda red homologous recombination. By replacing the corresponding genomic region by a chloramphenicol acetyl-transferase gene (cat) cassette, triplet clones were obtained and screened for MYC inhibitory activity (FIG. 5E). The delta 2 mutants failed to suppress MYC, suggesting that the gene responsible for bacterial MYC inhibition is located on this fragment (FIG. 5C). Further sequential deletions identified the delta 2.5.3 fragment as a particular determinant of MYC inhibition, (FIG. 5H).

The 2.5.3 DNA fragment (ORF42-ORF46) (SEQ ID NO 1) is located upstream of the a-hemolysin operon (see FIG. 8). It comprises bp numbers 3,989,295 to 3,992,678 on the E. coli 536 complete genome, or from 41,516 to 44, 899 bp on PAI I from 536.

In particular, the region contains genes encoding an ABC (ATP Binding Cassette) transporter complex, responsible for iron transport and ATP dependent-ion uptake (i.e. Mn²⁺ and Zn²⁺ transport). The ABC complex consists of an ATP-binding protein, a membrane protein and a periplasmic binding protein. In addition, ORF44 showed significant sequence homology to the IS630 transposase.

The position of open reading frames present in the deleted chromosomal region and their corresponding proteins are summarized as follows:
48-227: ECP_3821 (hypothetical protein) (Part of ORF44 with similarity to IS630 transposase (CDS: ORF44)
430-1164: ECP 3822 (putative ABC metal-type transporter ATP-binding protein) (CDS: CAD33755.1)
1089-2015: ECP_3823 (putative ABC metal-type transporter permease)
2059-2937: ECP 3824 (putative metal ABC transporter substrate-binding protein) (CDS: CAD33756.1)
3124-3336: ECP_3825 (hypothetical protein-periplasmic binding protein (CDS: CAD33757.1))

Deduced protein sequences of the ABC transporter components are as follows:

- ECP_3823 (ZnuC family, ATP-binding cassette domain of ABC
metallic cations transporters):
(SEQ ID NO 2)
MDSYFLGGGTGMIALHSLAFGYSGQHPLGTLDGCFDTGSLTAIIGANGTGKSTLLKTLAG
LLPPLGGCFCMVPQGQRQLGYLPQLTEFDRQFPLSVNDLVLMGCIPHSGMFGRISCLWRK
KAIEALDTVGMTEFSQMHIGTLSGGQLQRVLFARLLVMQPSVILLDEPFTGIDVQTIRTL
LVVIRQLHLEGRTILAVLHDMEQVEKYFSHVLMLSAEGHRWGRSADILHSLTAAVTPPQQ
GLLP
- ECP_3824 (ZnuB family, metal ABC transporter substrate-
binding protein):
(SEQ ID NO 3)
MGEKCRYTPFPHGCSHATAAGAAAMMLLHLLCEPFGDEGEMRRALVGCLALTLSAAPLGC
FLLLRRMSLIGDALSHAVLPGVAIGYLVSGMSLVAMGVGGFIAGLSVAMLSGVVSRRTGL
REDASFAGFYLGSLALGVTLVSLRGSSVDLLHVLFGSILAIDANALITIGIISSGSVLVL
ALIYRVLVIESFDVTFLKVLSRRSRALIHCLFLSMVVLNLVAGFQLLGTLMTVGIMMLPA
ASARFWSQRLSIMLLAAVGIGTCASLVGLTWSYYADLPAGPAVILTSTLFFCFSVLEGSN
GGMLCRCR
ECP_3824 (ZnuA family, metal ABC transporter substrate-
binding protein):
(SEQ ID NO 4)
MKRSILVVALSSLLVSPLVIAKELNVVASFSVLGDMVSQIGGPYVHVTDLVQPDGDPHEF
EPSPKDSKTLAQADVVFVNGLGLEGWLDRLMKASGYRGEVITASNGIDTLKMKEDGTTIT
DPHAWNSMKNGIVYAHNIVNGLSKADPEHASDYRKOGDSYIQQLQQLDNYATQTFAAIPR
EKRKVLTSHDAFGYFAAAYGVRFLSPVGYSTESEASSKNVAKLINQIKREHVKLYFIENQ
TDPRLVKQIANASGAQAGGELYPEALTDSSGLAATYTAAFKHNVDTLAAGMK

Together with other ORFs on PAI I, these genes might encode a microRNA (miRNA), including a start- and a stop codon, and this miRNA may inhibit MYC.

### Example 7

### MYC Degradation by the Supernatant From E. coli 536

To address if bacteria produce MYC-degrading molecules, E. coli 536 was cultured in tissue culture medium under conditions, corresponding to those used for the cellular assays described in Example 5 above (RPMI for 4 hours). The supernatant was harvested by centrifugation and sterile filtered to remove remaining bacterial cells. Recombinant c-MYC protein was mixed with the supernatant and the change in MYC concentration was quantified by Western blot analysis (FIG. 5g).

A dose-dependent reduction in MYC content was detected, indicating that the supernatant was exerting a direct inhibitory effect.

### Example 8

### Mechanism of c-MYC Degradation in Infected Cells

a) CK1α1 Transcription factors are rapidly degraded and the half-life of MYC is typically around 30 minutes. The rapid loss of epithelial MYC staining suggested that bacteria accelerate MYC degradation and/or inhibit MYC expression. Direct effects of infection on MYC RNA levels were not detected by RT-PCR or gene expression analysis. Inhibitors of MYC-dependent gene networks were upregulated by E. coli 536 but not by the PAI I mutant, suggesting an effects at the level of transcription. Carbonyl-reductase and the DNA binding protein I, HLH were derepressed by the mutant strain.

A distinct set of MYC-related genes was differentially regulated in cells infected with E. coli 536 or the PAI I Δ2.5.3 mutant, however. Casein kinase 1α1 (CK1α1) expression was activated by E. coli 536 but not by 536ΔPAI I or PAI IA2.5.3 (FIG. 7C). CK1α1 phosphorylates MYC at Serine 252, marking the phosphorylated protein for proteasomal degradation. CK1α1 activation in infected cells was confirmed by confocal microscopy after staining with specific antibodies. A re-distribution of CK1α1 from the nucleus to cytoplasmic particles/vesicles characteristic of CK1α1 activation was detected in E. coli 536-infected cells. In addition, an increase in CK1α1 protein levels was demonstrated by western blot analysis.

In contrast, CK1α1 was not altered in cells infected with the 536ΔPAI I or PAIIΔ 2.5.3. MYC degradation by CK1α1 was supported by pharmacologic inhibition experiments. The CK1α1 inhibitor IC261 prevented MYC degradation in E. coli 536-infected cells but a Serine protease inhibitor (Pefabloc) had no effect (FIG. 7F). Consistent with these findings, we detected an increase in epithelial CK1α1 staining in vivo in C57BL/6 mice infected with E. coli 536 but not in mice infected with the 536ΔPAI I mutant (FIG. 7C, quantified in FIG. 7D).

In addition to CKα1, several genes involved in proteosomal degradation were differentially regulated, suggesting that infection activates proteasomal MYC degradation by a CK-1α1-dependant mechanism (FIG. 7A).

b) IFNβ. Like CK1α1, IFNβ suppresses MYC expression, by stimulating proteasomal MYC degradation. In addition, IFNβ inhibits MYC expression, by direct interference at the level of the MYC promoter. Epithelial IFNβ expression in cells infected with E. coli 536 or PAI I Δ2.5.3 was quantified (FIG. 7I). E. coli 536 triggered a rapid increase in IFNβ expression, accompanied by nuclear translocation after one hour, with a further increase after four hours. Consistent with these findings, we detected an increase in epithelial IFNβ staining in vivo in C57BL/6 mice infected with E. coli 536 but not in mice infected with the 536ΔPAI I mutant (data not shown).

The results identify a CK1α1- and IFNβ mechanism for MYC degradation, which is activated by E. coli 536 infection but not by the PAI I or Δ2.5.3 mutant.

TABLE 1
Transcriptional regulators activated in the primary ABU patients

| | Pr1 | | Pr6 | | Pr7a | | Pr7b | | Pr10 | | Pr16 | |
|---|---|---|---|---|---|---|---|---|---|---|---|---|
| | | p-value | | p-value | | p-value | | p-value | | p-value | | p-value |
| Transcription | | of | | of | | of | | of | | of | | of |
| Regulator | z-score | overlap | z-score | overlap | z-score | overlap | z-score | overlap | z-score | overlap | z-score | overlap |
| RUNX2 | 2.390 | 2.26E−02 | — | — | | | — | — | — | — | — | — |
| MYBL2 | — | — | 2 | 1.55E−03 | | | — | — | — | — | — | — |
| YY1 | — | — | 2.193 | 2.51E−02 | | | — | — | — | — | — | — |
| GLI2 | — | — | 2 | 1.37E−01 | | | — | — | — | — | — | — |
| IRF7 | — | — | — | — | 4.526 | 2.74E−11 | — | — | — | — | — | — |
| STAT2 | — | — | — | — | 2.195 | 9.87E−06 | — | — | — | — | — | — |
| IRF1 | — | — | — | — | 4.255 | 2.51E−05 | — | — | — | — | — | — |
| IRF3 | — | — | — | — | 2.263 | 1.02E−04 | — | — | — | — | — | — |
| Hdac | — | — | — | — | 2.065 | 3.72E−02 | — | — | — | — | — | — |
| NCOA1 | — | — | — | — | 2.193 | 3.92E−01 | — | — | — | — | — | — |
| NFKBIB | — | — | — | — | — | — | 2.236 | 1.24E−03 | — | — | — | — |
| BACH1 | — | — | — | — | — | — | — | — | 2.373 | 1.53E−03 | — | — |
| estrogen | — | — | — | — | — | — | — | — | — | — | 2 | 1.68E−02 |
| receptor | | | | | | | | | | | | |
| PPARG | — | — | — | — | — | — | — | — | — | — | 2.026 | 4.73E−02 |

TABLE 2
Transcription regulators activated in APN patients

| | APN6 | | APN7 | | APN10 | |
|---|---|---|---|---|---|---|
| Transcription | Regulation | p-value of | Regulation | p-value of | Regulation | p-value of |
| Regulator | z-score | overlap | z-score | overlap | z-score | overlap |
| MYCN | 4.703 | 5.66E−13 | 4.575 | 3.85E−13 | 5.127 | 1.16E−15 |
| MYC | 4.423 | 9.19E−04 | 3.934 | 3.77E−04 | 4.087 | 5.45E−09 |
| KLF2 | 3.128 | 2.11E−01 | 2.599 | 3.69E−01 | 3.527 | 4.10E−01 |
| NCOR2 | 2.033 | 2.78E−01 | — | | 2.029 | 6.25E−02 |
| WT1 | 2.209 | 9.06E−02 | — | | — | — |
| ESR1 | 2.006 | 5.13E−01 | — | | — | — |
| CIITA | — | — | 2.31 | 1.41E−01 | — | |
| CUX1 | — | — | 2.068 | 1.06E−01 | — | — |

1. A pharmaceutical composition comprising a bacteria which expresses an inhibitor of MYC activity or a extract or product obtainable therefrom, and at least one pharmaceutically acceptable carrier.

2. The pharmaceutical composition according to claim 1 which comprises a bacteria.

3. The pharmaceutical composition according to claim 1 which comprises a supernatant obtainable by culture of a bacteria.

4. The pharmaceutical composition according to claim 1 which comprises a factor isolated from a bacteria, which inhibits MYC activity.

5. The pharmaceutical composition according to claim 1 wherein the bacteria is an attenuated uropathogenic bacteria, or a recombinant bacteria comprising an asymptomatic bacteriuria (ABU) strain which has been engineered to express an inhibitor of MYC activity obtainable from a uropathogenic strain.

6. The pharmaceutical composition according to claim 5 wherein the attenuated uropathogenic bacteria is an E. coli strain.

7. The pharmaceutical composition according to claim 6 wherein the bacteria is selected from CFT073 and 536E.

8-9. (canceled)

10. A pharmaceutical composition comprising an inhibitor of MYC activity comprising a factor obtainable from a bacteria, and at least one pharmaceutically acceptable carrier.

11. A pharmaceutical composition according to claim 10 wherein the bacteria is a uropathogenic E. coli.

12. A pharmaceutical composition according to claim 11 wherein the uropathogenic E. coli is selected from CFT073 and 536.

13. A pharmaceutical composition according to claim 10 wherein the inhibitor of MYC activity comprises a protein or RNA expressed by a gene found in a PAI I or PAI II region of the genome of said bacteria.

14. A pharmaceutical composition according to claim 13 wherein the gene is a gene found in the PAI I region of the genome.

15. A pharmaceutical composition according to claim 14 wherein the PAI I region is represented by SEQ ID NO 1.

16. A pharmaceutical composition according to claim 10 wherein the inhibitor of MYC activity comprises a bacterial ABC transporter protein, a periplasmic binding protein, or an active fragment of either of these.

17-19. (canceled)

20. A method of treating or preventing a disease in which MYC levels are elevated, said method comprising administering to a patient in need thereof, an effective amount of the pharmaceutical composition according to claim 1.

21. The method according to claim 20, wherein the disease is cancer.

22. The method according to claim 20, wherein the cancer is an epithelial or mucosal cancer.

23. A method of treating or preventing a disease in which MYC levels are elevated, said method comprising administering to a patient in need thereof, an effective amount of the pharmaceutical composition according to claim 10.

24. The method according to claim 23, wherein the disease is cancer.

25. The method according to claim 23, wherein the cancer is an epithelial or mucosal cancer.


SEQUENCE LISTING (4 sequences)

SEQ ID NO: 1 - DNA, 3384 bp, Escherichia coli
  misc_feature 1..3384: note = Genomic sequence
  source 1..3384: mol_type = genomic DNA
atcagcatggcagtcagtctgcgggcataatttttatcgtgtgttttatggatggctttctgcatcaggtgtcgttcgtcacgggaaattggtgctatgatcggcattgctcagttcggttggtgatttgttttgatttggcgattgatcagagcgcacaattcgggctgagtaccctcaaagtgatctactattccgcgcagctatttagcgcattacactgctaaatgactaacgcatccctgtcaatactgctctcgctgtatgcttacgctgcagaagccatgacacattttcttacctatcatcggcacccataaagcaacagtgttatactataacaattttgatgggggatactgtggtcagtctttatgctttttccgcaatagcccgactgggtatcgtcggtgtgctgctggtactgttatggactcttatttcttgggcggtggtactggcatgatcgcattacattcactggcatttggttattcgggacagcacccgctgggtacgctggatggatgttttgacaccggttcactgactgccattatcggagcgaatggcacaggtaagtccaccttgcttaaaacgttggccggtctgctgccacccctggggggttgcttctgtatggtaccgcaggggcaacgccagttaggttatttgccccaattgactgaatttgatcgccagtttccactgagtgttaacgatctggtgctgatgggatgtattccacacagcgggatgttcggtcgtatttcctgtctatggcgaaaaaaagctatcgaagcgctggataccgtcggtatgacagaattttcgcagatgcacatcggcacactttccggtggtcagttacagcgcgtactgttcgcacgactgctggtgatgcagccgtcggttattctgctggatgaaccatttactggcattgatgttcagactatccgtactctgctggttgttattcggcagctacatctggaaggacgtacaattcttgcggtgttgcatgacatggaacaggtagaaaaatacttttctcatgtactgatgctcagtgcggaaggccaccgatgggggagaagtgccgatatactccattccctcacggctgcagtcacgccaccgcagcaggggctgctgccatgatgttactgcacctgttgtgtgagccatttggcgatttcggttttatgcgtcgggcactggtcggatgtctggcgctgaccctgagtgccgcgccacttggctgttttttgctactgcgccgtatgagcctgatcggtgatgcactgtcccatgcggtgttgccgggagtagcaattggctacctggtatcgggcatgtcgctggttgcgatgggagtcggtggcttcattgccgggctgtctgtggcgatgttgtctggtgtggtcagccgccgcactggattgagagaagacgccagtttcgctgggttttacctcggctcgctggcacttggcgtcaccctggtctctttgcgtggctccagtgtcgacctgctgcatgtgctgttcggttccattctggctattgacgccaatgctctgatcactatcggcataattagttccggttcagttctggtactggcgttgatctaccgggtgctggtgattgagtcgtttgatgtcactttcctgaaagttctgtcacgtcggtcgcgggcgctgatccactgcctgtttttgtcaatggtggtactcaatctggtggctggtttccagctacttggcaccctgatgacagtcggtattatgatgttaccggctgccagcgcgcgtttctggagtcaacgtctgtccatcatgttgttagcggcagtgggtattggtacctgcgccagtctggttggactgacctggtcttattacgctgatttgcctgcaggccctgcggtcatcctcacgagcactctgtttttctgcttttcagttctgtttggctctaatggcggcatgttatgcagatgtcgctgacaagaagtgtttttacacatagcaacgagatgaaggaggaaagatgaaacggtccattttggttgtggcactgtccagcctactagtaagcccactggtgattgcaaaagagttgaatgtggtggcaagtttctcggtactgggcgatatggtcagccagattggcggcccatacgttcatgtcactgatctggtgcaaccggatggtgatccacatgaatttgaaccgtcgccgaaagacagcaaaacgctggcacaggcggatgttgtctttgtcaatggattgggactggagggatggctggaccgtctgatgaaagcctctggctacagaggtgaggtaattaccgcgtctaacggaatagatacactgaaaatgaaagaggatgggaccaccatcactgatccgcacgcctggaacagcatgaaaaacggtattgtctatgctcacaacattgtcaatggcctgagcaaagcagacccggagcacgccagcgattatcgaaaacagggtgacagctatatccagcagttgcagcaactggataattacgccacgcagacgtttgctgccattccaagagaaaaacgtaaagtactgaccagtcatgatgcgtttgggtatttcgctgccgcctacggggttcgctttctgtcgccagtggggtactccactgagtcggaagctagcagtaaaaatgtggcgaaactgattaatcaaattaagagagagcacgtaaaattgtactttatcgaaaaccagaccgatccccgtttggtgaaacaaatcgccaatgccagtggtgcacaagcgggaggagagctttatccggaagcacttaccgacagtagcggcctcgctgctacctacacggcagcgttcaaacacaatgtcgataccctcgctgctggcatgaagtaagagagttggcaaaaaagtaagtagatatcaggttgccccatatcatcaggtaacctgtgacacttctcttgccagagcgccatgttccagcgcctcactcactatctgcttatcatccgactttccctcatacctgtctcgattataaagataaaacgatgatgcccgggttcattgtgcgaaggcatggcatatttgttcccggtgtcgtcagacagcatcattcgactctccatctgctgtctgaccagacaaaagatggccttgtttgccgcggtggaaatggagtgccgtctgttctttatttattgtttatgttatgttatagcatataaaagagtattgtttggcatctgtaacttattgagaaggcaaactgcagagtggttaatgcagtaattgtattagctaaaattttgttaatcaaaatttgtttgattgttaatg

SEQ ID NO: 2 - protein, 244 aa, Escherichia coli
  REGION 1..244: note = ABC transporter protein
MDSYFLGGGTGMIALHSLAFGYSGQHPLGTLDGCFDTGSLTAIIGANGTGKSTLLKTLAGLLPPLGGCFCMVPQGQRQLGYLPQLTEFDRQFPLSVNDLVLMGCIPHSGMFGRISCLWRKKAIEALDTVGMTEFSQMHIGTLSGGQLQRVLFARLLVMQPSVILLDEPFTGIDVQTIRTLLVVIRQLHLEGRTILAVLHDMEQVEKYFSHVLMLSAEGHRWGRSADILHSLTAAVTPPQQGLLP

SEQ ID NO: 3 - protein, 308 aa, Escherichia coli
  REGION 1..308: note = ABC transporter protein
MGEKCRYTPFPHGCSHATAAGAAAMMLLHLLCEPFGDFGFMRRALVGCLALTLSAAPLGCFLLLRRMSLIGDALSHAVLPGVAIGYLVSGMSLVAMGVGGFIAGLSVAMLSGVVSRRTGLREDASFAGFYLGSLALGVTLVSLRGSSVDLLHVLFGSILAIDANALITIGIISSGSVLVLALIYRVLVIESFDVTFLKVLSRRSRALIHCLFLSMVVLNLVAGFQLLGTLMTVGIMMLPAASARFWSQRLSIMLLAAVGIGTCASLVGLTWSYYADLPAGPAVILTSTLFFCFSVLFGSNGGMLCRCR

SEQ ID NO: 4 - protein, 292 aa, Escherichia coli
  REGION 1..292: note = ABC transporter protein
MKRSILVVALSSLLVSPLVIAKELNVVASFSVLGDMVSQIGGPYVHVTDLVQPDGDPHEFEPSPKDSKTLAQADVVFVNGLGLEGWLDRLMKASGYRGEVITASNGIDTLKMKEDGTTITDPHAWNSMKNGIVYAHNIVNGLSKADPEHASDYRKQGDSYIQQLQQLDNYATQTFAAIPREKRKVLTSHDAFGYFAAAYGVRFLSPVGYSTESEASSKNVAKLINQIKREHVKLYFIENQTDPRLVKQIANASGAQAGGELYPEALTDSSGLAATYTAAFKHNVDTLAAGMK